(19) United States
(12) Patent Application Publication
(10) Pub. No.: US 2011/0178054 A1
(43) Pub. Date: Jul. 21, 2011

(54) Heterocyclic GPCR Agonists

(75) Inventors: Lisa Sarah Bertram, Oxford (GB); Matthew Colin Thor Fyfe, Oxford (GB); William Gattrell, Oxford (GB); Revathy P. Jeevaratnam, Oxford (GB); John Keily, Oxford (GB); Martin James Procter, Oxford (GB)

(73) Assignee: Prosidion Limited, Oxford (GB)

(21) Appl. No.: 13/003,573

(22) PCT Filed: Jul. 10, 2009
(86) PCT No.: PCT/GB09/50831

§ 371 Date: Apr. 11, 2011

(30) Foreign Application Priority Data
Jul. 10, 2008 (GB) 0812649.2

(51) Int. Cl. A61K 31/454; C07D 413/04; C07D 413/14; A61K 31/5377; A61P 3/04; A61P 3/10; A61P 3/00; A61P 3/06; A61P 9/12

(52) U.S. Cl. 514/210.2; 546/209; 544/130; 514/326; 514/236.2

(57) ABSTRACT

Compounds of formula (I): or pharmaceutically acceptable salts thereof, are GPCR (GPR119) agonists and are useful as for the treatment of diabetes and obesity.

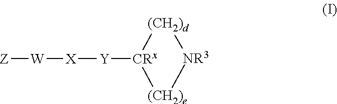

### BACKGROUND OF THE INVENTION

The present invention is directed to G-protein coupled receptor (GPCR) agonists. In particular, the present invention is directed to agonists of GPR119 that are useful for the treatment of obesity, e.g. as regulators of satiety, metabolic syndrome and for the treatment of diabetes.

Obesity is characterized by an excessive adipose tissue mass relative to body size. Clinically, body fat mass is estimated by the body mass index (BMI; weight (kg)/height (m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent applications WO2005/061489, WO2006/070208 and WO2006/067532 disclose heterocyclic derivatives as GPR119 receptor agonists. International patent applications WO2006/067531, WO2007/003960, WO2007/003961, WO2007/003962 and WO2007/003964, WO2007/116230 and WO2007/116229 disclose GPR119 receptor agonists.

The present invention relates to agonists of GPR119 which are useful for the treatment of diabetes and as peripheral regulators of satiety, e.g. for the treatment of obesity and metabolic syndrome.

### SUMMARY OF THE INVENTION

Compounds of formula (I):

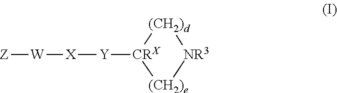

or pharmaceutically acceptable salts thereof, are agonists of GPR119 and are useful for the prophylactic or therapeutic treatment of diabetes and obesity.

### DETAILED DESCRIPTION OF THE INVENTION

The present invention is directed to a compound of formula (I), or a pharmaceutically acceptable salt thereof:

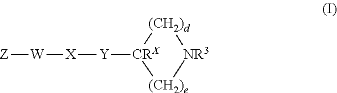

wherein Z is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR¹, CN, NO₂, —(CH₂)ⱼ—S(O)ₘR¹, —(CH₂)ⱼ—C(O)NR¹R¹¹, NR¹R¹¹, NR²C(O)R¹, NR²C(O)NR¹R¹¹, NR²SO₂R¹, SO₂NR¹R¹¹, C(O)R², C(O)OR², —P(O)(CH₃)₂, —(CH₂)ⱼ-(4- to 7-membered heterocyclyl) or —(CH₂)ⱼ-(5- to 6-membered heteroaryl);

m is 0, 1 or 2;

j is 0, 1 or 2;

W and Y are independently a bond, an unbranched or a branched C₁₋₄ alkylene optionally substituted by hydroxy or C₁₋₃alkoxy, or an unbranched or a branched C₂₋₄ alkenylene;

X is selected from CH₂, O, S, CH(OH), CH(halogen), CF₂, C(O), C(O)O, C(O)S, SC(O), C(O)CH₂S, C(O)CH₂C(OH), C(OH)CH₂C(O), C(O)CH₂C(O), OC(O), NR⁵, CH(NR⁵R⁵⁵), C(O)NR², NR²C(O), S(O) and S(O)₂;

R^{x} is hydrogen or hydroxy;

R¹ and R¹¹ are independently hydrogen, C₁₋₅ alkyl, which may optionally be substituted by halo, hydroxy, C₁₋₄ alkoxy-, aryloxy-, arylC₁₋₄alkoxy-, C₁₋₄ alkylS(O)ₘ—, C₃₋₇ heterocyclyl, —C(O)OR⁷ or N(R²)₂; or may be C₃₋₇ cycloalkyl or heterocyclyl, wherein the cyclic groups may be substituted with one or more substituents selected from halo, C₁₋₄ alkyl, C₁₋₄ fluoroalkyl, OR⁶, CN, SO₂CH₃, CH₂OH, N(R²)₂ and NO₂; or taken together R¹ and R¹¹ may form a 5- or 6-membered heterocyclic ring optionally substituted by hydroxy, C₁₋₄ alkyl, C₁₋₄ hydroxyalkyl, or CH₂NH₂ and optionally containing a further heteroatom selected from O and NR²; or R¹¹ is C₁₋₄ alkyloxy-;

R² are independently hydrogen or C₁₋₄ alkyl; or a group N(R²)₂ may form a 4- to 7-membered heterocyclic ring optionally containing a further heteroatom selected from O and NR²;

R³ is:

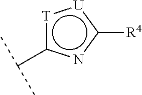

wherein one of T and U is O and the other is N;

R⁴ is C₁₋₃ hydroxyalkyl, C₁₋₃ alkoxyC₁₋₃ alkyl, C₁₋₃ fluoroalkyl, —(C₁₋₃ alkylene)ₖ-N(R⁶)₂, —(C₁₋₃ alkylene)ₖ-C₃₋₆ cycloalkyl or —(C₁₋₃ alkylene)ₖ-4- to 6-membered heterocyclyl where the cycloalkyl and heterocyclyl groups may be optionally substituted with one or more C₁₋₃ alkyl or fluorine groups;

k is 0 or 1;

R⁵ and R⁵⁵ are independently hydrogen or C₁₋₄ alkyl; or taken together R⁵ and R⁵⁵ may form a 5- or 6-membered heterocyclic ring; or a group NR⁵ may represent NS(O)₂-(2-NO₂—C₆H₄);

R⁶ are independently selected from hydrogen and C₁₋₃ alkyl;

R⁷ is hydrogen or C₁₋₄ alkyl;

d is 0, 1, 2 or 3; and

e is 1, 2, 3, 4 or 5, provided that d+e is 2, 3, 4 or 5.

The molecular weight of the compounds of formula (I) is preferably less than 800, more preferably less than 600, even more preferably less than 500.

Suitably Z represents phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR¹, CN, NO₂, S(O)ₘR¹, C(O)NR¹R¹¹, NR¹R¹¹, NR²C(O)R¹, NR²SO₂R¹, SO₂NR¹R¹¹, COR², C(O)OR², a 4- to 7-membered heterocyclyl group or a 5- or 6-membered heteroaryl group. More suitably Z represents phenyl or a 6-membered heteroaryl group containing up to four N atoms.

In one embodiment of the invention Z is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR¹, CN, NO₂, S(O)ₘR¹, C(O)NR¹R¹¹, NR¹R¹¹, NR²C(O)R¹, NR²SO₂R¹, SO₂NR¹R¹¹, C(O)R², C(O)OR², 4- to 7-membered heterocyclyl or 5- to 6-membered heteroaryl.

Z is preferably phenyl or a 6-membered heteroaryl group containing up to two N heteroatoms, e.g. 2-pyridyl, either of which may optionally be substituted, more preferably optionally substituted phenyl or 2-pyridyl and especially substituted phenyl. Examples of Z heteroaryl groups include oxazolyl, isoxazolyl, thienyl, pyrazolyl, imidazolyl, furanyl, pyridazinyl or 2-pyridyl. Preferred substituent groups for Z are halo, C₁₋₄ alkyl, C₁₋₄ fluoroalkyl, C₂₋₄ alkenyl, C₂₋₄ alkynyl, CN, S(O)ₘR¹, NR²C(O)NR¹R¹¹, C(O)NR¹R¹¹, SO₂NR¹R¹¹, COR², COOR² or a 5- or 6-membered heteroaryl group; especially halo e.g. fluoro or chloro, C₁₋₄ alkyl, C₁₋₄ fluoroalkyl, C₂₋₄ alkenyl, C₂₋₄ alkynyl, CN, S(O)ₘR¹, NR²C(O)NR¹R¹¹, C(O)NR¹R¹¹, SO₂NR¹R¹¹ or a 5-membered heteroaryl group; in particular fluoro, chloro, methyl, S(O)ₘR¹ e.g. where m is 1 or 2, NR²C(O)NR¹R¹¹, C(O)NR¹R¹¹, SO₂NR¹R¹¹ or a 5-membered heteroaryl group.

In one embodiment, suitable substituent groups for Z are halo, C₁₋₄ alkyl, C₁₋₄ fluoroalkyl, C₂₋₄ alkenyl, C₂₋₄ alkynyl, CN, S(O)ₘR¹, C(O)NR¹R¹¹, SO₂NR¹R¹¹, COR², COOR² or a 5- or 6-membered heteroaryl group; especially halo (e.g. fluoro or chloro), C₁₋₄ alkyl, C₁₋₄ fluoroalkyl, C₂₋₄ alkenyl, C₂₋₄ alkynyl, CN, S(O)ₘR¹, C(O)NR¹R¹¹, SO₂NR¹R¹¹; in particular fluoro, chloro, methyl, S(O)ₘR¹ (e.g. where m is 1 or 2), C(O)NR¹R¹¹ or SO₂NR¹R¹¹. Specific Z groups which may be mentioned are those where Z is phenyl substituted by —SO₂Me or —CONHR^{d}, preferably —CONHR^{d}, wherein R^{d} is hydrogen, 5-membered heterocyclyl, C₁₋₃ alkyl, or C₂₋₃ alkyl substituted by amino and/or one or two hydroxy groups, and wherein Z is optionally additionally substituted by one or two methyl groups. The —SO₂Me or —CONHR^{d} substituent is preferably in the para position.

Suitably, j is 0 or 1. In one embodiment of the invention j represents 0. In a second embodiment of the invention j represents 1.

Suitably W and Y are independently a bond, an unbranched or a branched C₁₋₄ alkylene optionally substituted by hydroxy, or an unbranched or a branched C₂₋₄ alkenylene.

In one embodiment of the invention W and Y are independently a bond, an unbranched or a branched C₁₋₄ alkylene, or an unbranched or a branched C₂₋₄ alkenylene.

Preferably W and Y do not both represent a bond.

Preferably W is a bond.

Preferably Y is unbranched or a branched C₃₋₄ alkylene optionally substituted by hydroxy or C₁₋₃ alkoxy, e.g an unsubstituted unbranched or a branched C₃₋₄ alkylene.

In certain embodiments of the invention —W—X—Y— represents a chain of 2 to 6 atoms in length. —W—X—Y— preferably represents a 4 or 5 atom chain.

When W is C₂₋₃ alkenylene, the stereochemistry at the double bond is preferably (E).

Suitably, X is selected from CH₂, O, S, CH(OH), CH(halogen), CF₂, C(O), C(O)O, C(O)S, SC(O), C(O)CH₂S, C(O)CH₂C(OH), C(O)CH₂C(O), OC(O), NR⁵, CH(NR⁵R⁵⁵), C(O)NR², S(O) and S(O)₂. More suitably X is selected from CH₂, O, S, CH(OH), CH(halogen), C(O), C(O)O, C(O)S, SC(O), C(O)CH₂S, C(O)CH₂C(OH), C(O)CH₂C(O), OC(O), NR⁵, CH(NR⁵R⁵⁵), C(O)NR², S(O) and S(O)₂.

X is preferably CH₂, CF₂, O or NR⁵ e.g. NH, in particular CH₂, O or NR⁵, especially O.

A preferred group represented by —W—X—Y— is —O—CH₂—CH₂—CR^{y}—, where R^{y} is hydrogen or methyl.

R^{x} is preferably hydrogen.

Suitably, R¹ and R¹¹ are independently hydrogen, C₁₋₄ alkyl, which may optionally be substituted by halo e.g. fluoro, hydroxy, C₁₋₄ alkyloxy-, aryloxy-, arylC₁₋₄ alkyloxy-, C₁₋₄ alkylS(O)ₘ—, C₃₋₇ heterocyclyl or N(R²)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁶, CN, SO₂CH₃, N(R²)₂ and NO₂; or taken together R¹ and R¹¹ may form a 5- or 6-membered heterocyclic ring optionally containing a further heteroatom selected from O and NR².

In one embodiment of the invention R¹ and R¹¹ are independently hydrogen, C₁₋₄ alkyl, which may optionally be substituted by halo (e.g. fluoro), hydroxy, C₁₋₄ alkyloxy-, C₁₋₄ alkylthio-C₃₋₇ heterocyclyl or N(R²)₂; or may be C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁶, CN, SO₂CH₃, N(R²)₂ and NO₂.

Suitably R² is hydrogen, methyl or tert-butyl.

T is preferably O and U is preferably N.

Exemplary R⁴ groups include those provided in the examples.

A group of compounds which may be mentioned are those where R⁴ is C₃₋₆ cycloalkyl, C₁₋₃ hydroxyalkyl, C₁₋₃ alkoxyC₁₋₃ alkyl, —(CH₂)ₖ—N(R⁶)₂, or —(CH₂)ₖ-5- to 6-membered heterocyclyl which heterocyclyl group may be optionally substituted with C₁₋₃ alkyl; and k is 0 or 1.

In one embodiment of the invention d+e is 2, 3, or 4. Suitably, d is 1 or 2 and e is 1 or 2. In a preferred embodiment of the invention d and e each represent 1. In a more preferred embodiment of the invention d and e each represent 2.

Suitably R⁵ and R⁵⁵ are independently hydrogen or C₁₋₄alkyl; or taken together R⁵ and R⁵⁵ may form a 5- or 6-membered heterocyclic ring; in particular R⁵ represents hydrogen or methyl, especially methyl.

Suitably R⁶ is C₁₋₄ alkyl.

A preferred group of compounds of are those of formula (Ia), and pharmaceutically acceptable salts thereof:

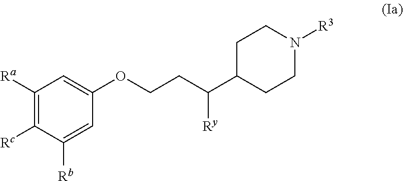

wherein:

R³ is as described previously for compounds of formula (I);

R^{y} is hydrogen or methyl;

R^{a} and R^{b} are independently selected from hydrogen and methyl;

R^{c} is —SO₂Me or —CONHR^{d};

R^{d} is hydrogen, 5-membered heterocyclyl, C₁₋₃ alkyl, or C₂₋₃ alkyl substituted by amino or one or two hydroxy groups.

In one embodiment of the compounds of formula (Ia), R^{c} is —SO₂Me, in another, R^{c} is —CONHR^{d}.

R^{c} is preferably —CONHR^{d}.

One or both of R^{a} and R^{b} are preferably methyl, more preferably R^{a} is methyl and R^{b} is hydrogen.

In one embodiment of the compounds of formula (Ia) R^{y} is hydrogen and in another R^{y} is methyl. R^{y} is preferably hydrogen. When R^{y} is methyl, the stereocentre produced preferably has the (R)-configuration.

R^{d} is preferably hydrogen or C₂₋₃alkyl substituted by one or two hydroxy groups. R⁶ is more preferably C₂₋₃alkyl substituted by one or two hydroxy groups, e.g. 2-hydroxyethyl, 2-hydroxy-1-methylethyl, 2,3-dihydroxypropyl or 2-hydroxy-1-hydroxymethylethyl.

A group of compounds which may be mentioned are those of formula (Ib) and pharmaceutically acceptable salts thereof:

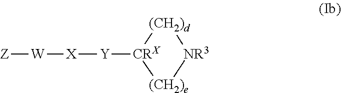

wherein Z is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR¹, CN, NO₂, —(CH₂), —S(O)ₘR¹, —(CH₂), —C(O)NR¹R¹¹, NR¹R¹¹, NR²C(O)R¹, NR²C(O)NR¹R¹¹, NR²SO₂R¹, SO₂NR¹R¹¹, C(O)R², C(O)OR², —P(O)(CH₃)₂, —(CH₂)ⱼ-(4- to 7-membered heterocyclyl) or —(CH₂)ⱼ-(5- to 6-membered heteroaryl);

m is 0, 1 or 2;

j is 0, 1 or 2;

W and Y are independently a bond, an unbranched or a branched C₁₋₄ alkylene optionally substituted by hydroxy or C₁₋₃alkoxy, or an unbranched or a branched C₂₋₄ alkenylene;

X is selected from CH₂, O, S, CH(OH), CH(halogen), CF₂, C(O), C(O)O, C(O)S, SC(O), C(O)CH₂S, C(O)CH₂C(OH), C(OH)CH₂C(O), C(O)CH₂C(O), OC(O), NR⁵, CH(NR⁵R⁵⁵), C(O)NR², NR²C(O), S(O) and S(O)₂;

R^{x} is hydrogen or hydroxy;

R¹ and R¹¹ are independently hydrogen, C₁₋₄ alkyl, which may optionally be substituted by halo, hydroxy, C₁₋₄ alkoxy-, aryloxy-, arylC₁₋₄ alkoxy-, C₁₋₄ alkylS(O)ₘ—, C₃₋₇ heterocyclyl, —C(O)OR⁷ or N(R²)₂; or may be C₃₋₇ cycloalkyl or heterocyclyl, wherein the cyclic groups may be substituted with one or more substituents selected from halo, C₁₋₄ alkyl, C₁₋₄ fluoroalkyl, OR⁶, CN, SO₂CH₃, N(R²)₂ and NO₂; or taken together R¹ and R¹¹ may form a 5- or 6-membered heterocyclic ring optionally substituted by hydroxy, C₁₋₄ alkyl or C₁₋₄ hydroxyalkyl and optionally containing a further heteroatom selected from O and NR²; or R¹¹ is C₁₋₄ alkyloxy-;

R² are independently hydrogen or C₁₋₄ alkyl; or a group N(R²)₂ may form a 4- to 7-membered heterocyclic ring optionally containing a further heteroatom selected from O and NR²;

R³ is:

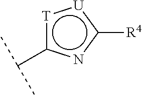

wherein one of T and U is O and the other is N;

R⁴ is C₃₋₆ cycloalkyl, C₁₋₃ hydroxyalkyl, C₁₋₃ alkoxyC₁₋₃ alkyl, —(CH₂)ₖ—N(R⁶)₂, or —(CH₂)ₖ-5- to 6-membered heterocyclyl which heterocyclyl group may be optionally substituted with C₁₋₃ alkyl;

k is 0 or 1;

R⁵ and R⁵⁵ are independently hydrogen or C₁₋₄alkyl; or taken together R⁵ and R⁵⁵ may form a 5- or 6-membered heterocyclic ring; or a group NR⁵ may represent NS(O)₂-(2-NO₂—C₆H₄);

R⁶ are independently selected from hydrogen and C₁₋₃ alkyl;

R⁷ is hydrogen or C₁₋₄ alkyl;

d is 0, 1, 2 or 3; and

e is 1, 2, 3, 4 or 5, provided that d+e is 2, 3, 4 or 5.

As used herein, unless stated otherwise, “alkyl” as well as other groups having the prefix “alk” such as, for example, alkylene, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The term “cycloalkyl” means carbocycles containing no heteroatoms, and includes monocyclic and bicyclic saturated and partially saturated carbocycles. Examples of cycloalkyl groups include cyclopropyl, cyclobutyl, cyclopentyl, cyclohexyl and cycloheptyl. Examples of partially saturated cycloalkyl groups include cyclohexene and indane. Cycloalkyl groups will typically contain 3 to 10 ring carbon atoms in total (e.g. 3 to 6, or 8 to 10).

The term “halo” includes fluorine, chlorine, bromine, and iodine atoms (in particular fluorine or chlorine).

The term “aryl” includes phenyl and naphthyl, in particular phenyl. Unless otherwise indicated the term “heterocyclyl” and “heterocyclic ring” includes 4- to 10-membered monocyclic and bicyclic saturated rings, e.g. 4- to 7-membered monocyclic saturated rings, containing up to three heteroatoms selected from N, O and S. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered, e.g. monocyclic 5- or 6-membered, heteroaryl rings containing up to 4 heteroatoms selected from N, O and S. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine. Preferred heteroaryl groups are monocyclic 5- or 6-membered, heteroaryl rings containing up to 4 heteroatoms selected from N, O and S.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, in which Z, d, e, W, X and Y are as defined above and G represents NR³. The Schemes are illustrated using compounds wherein R^{x} is hydrogen, compounds wherein R^{x} is hydroxy may be prepared using analogous methods.

Compounds of formula (I) in which X is CO₂, COS, or CONR² can be prepared by condensing the appropriate acid (II) with an alcohol, thiol, or amine (III), as shown in Scheme 1 where E is O, S, or NR², using a typical reagent for such a condensation reaction, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The acids (II) and alcohols, thiols, and amines (III) are either commercially available or are prepared easily using known techniques.

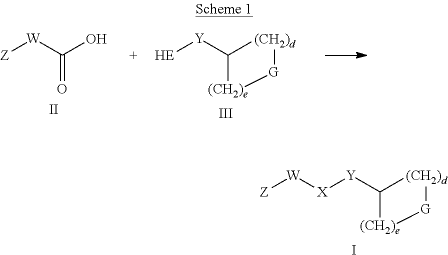

Compounds of formula (I) in which X is SCO or OCO can be prepared by condensing the appropriate thiol or alcohol (IV) with the appropriate acid (V), as shown in Scheme 2 where E is S or O, employing a reagent typically used for effecting such reactions, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The alcohols and thiols (IV), as well as acids (V), are either commercially available or are prepared straightforwardly using known techniques.

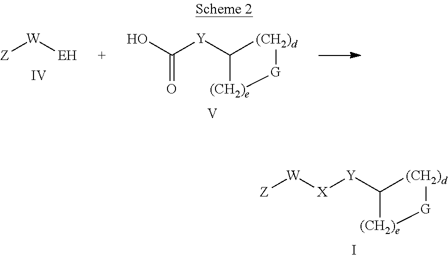

Compounds of formula (I) in which X is S or O can be prepared by alkylating the appropriate thiol or alcohol (IV) with the appropriate alkyl halide or sulfonate ester (VI), as shown in Scheme 3 where E is S or O and LG is chloro, bromo, iodo, alkanesulfonate, or arenesulfonate. The reaction is typically carried out using a base, e.g., potassium tert-butoxide (Hall, S. E., et al. J. Med. Chem. 1989, 32, 974-984). The alcohols and thiols (IV), as well as the alkyl halides or sulfonates (VI), are either commercially available or are made easily using known techniques. The compounds of formula (I) where X is SO or SO₂ can easily be obtained from the compounds of formula (I) where X is S by oxidation with, for example, mCPBA (Fyfe, M. C. T. et al. International Patent Publication WO 04/72031).

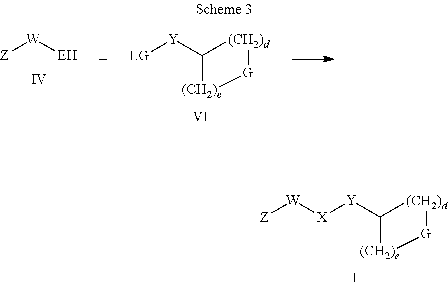

Compounds of formula (I) in which W is C₂₋₃ alkenylene can be prepared by a Wittig reaction between the appropriate phosphonium salt (VII) and the appropriate aldehyde (VIII), as indicated in Scheme 4 where m is 1 or 2 and n is 0 or 1 with the proviso that m+n<3. As an alternative, to the approach described in Scheme 4, the compounds of formula (I) in which W is C₂₋₃ alkenylene can be prepared by a Wittig reaction between the appropriate aldehyde (IX) and the appropriate phosphonium salt (X), as indicated in Scheme 5 where q is 0 or 1 and r is 1 or 2 with the proviso that q+r<3. The reactions are carried out in the presence of a suitable base, e.g., NaOMe or LiHMDS (March, J. Advanced Organic Chemistry, 4th edn.; Wiley: New York, 1992; pp 956-963). The phosphonium salts (VII) and (X), as well as the aldehydes (VIII) and (IX), are either commercially available or are made easily using known techniques. The compounds of formula (I) where W is C₂₋₃ alkylene can easily be synthesized from the compounds of formula (I) where W is C₂₋₃ alkenylene by a hydrogenation reaction using, for example, palladium on charcoal as a catalyst.

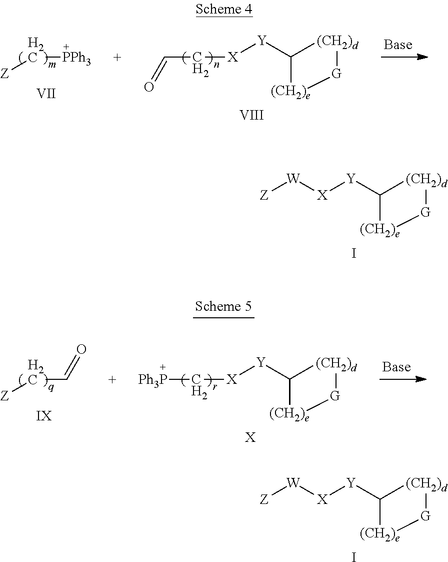

Compounds of the formula (I) where W is a bond, X is S or O, and the group Z is unsubstituted or substituted by CN can be prepared by condensation of the appropriate heteroaryl halide (XI), where with the appropriate alcohol or thiol (III), as depicted in Scheme 6 where Hal represents a halogen and E is S or O. The reaction is carried out in the presence of a suitable basic system, e.g., potassium hydroxide and potassium carbonate in the presence of tris(3,6-dioxaheptyl)amine (Ballesteros, P.; Claramunt, R. M.; Elguero, J. Tetrahedron 1987, 43, 2557-2564). The heteroaryl halides (XI) and alcohols/thiols (III) are either commercially available or are made easily using known techniques.

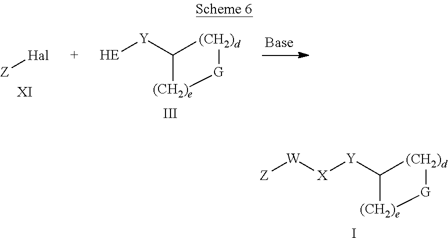

Compounds of the formula (I) may be prepared by condensation of amine (XII) with a heteroaryl chloride of formula (XIV), as illustrated in Scheme 7 (Barillari, C. et al. Eur. J. Org. Chem. 2001, 4737-4741; Birch, A. M. et al. J. Med. Chem. 1999, 42, 3342-3355).

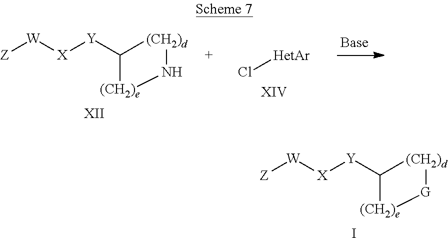

Compounds of the formula (I) where the group Z is substituted by CN can be prepared from the corresponding unsubstituted Z group by the Reissert reaction (Fife, W. K. J. Org. Chem. 1983, 48, 1375-1377). Similar reactions can be used to prepare the compounds where Z is substituted by halogen (Walters, M. A.; Shay, J. J. Tetrahedron Lett. 1995, 36, 7575-7578). The compounds where Z is substituted by halogen can be transformed into the corresponding compounds where Z is substituted by C₁₋₄ alkyl by transition metal-catalysed cross-coupling reactions (Fürstner, A., et al. J. Am. Chem. Soc. 2002, 124, 13856-13863).

The oxadiazole rings of the compounds of formula (I) may be prepared by the routes shown in Scheme 8 and using methods reviewed recently (Curr. Org. Chem. 2008, 12, 850-898). For example, treatment of amines of formula (XII) with cyanogen bromide followed by condensation of the resultant cyanamide (XV) with a compound of formula (XVIII) under standard conditions yields compounds of formula (I) where T is O and U is N. Compounds of formula (XVIII) are either commercially available, or readily prepared from the corresponding carboxylic acids or nitriles using well known techniques. Alternatively, synthesis of the regioisomeric oxadiazole, where T is N and U is O, can be achieved by heating compounds of formula (XV) with hydroxylamine to give N-hydroxyguanidines of formula (XVI) that may be condensed with a carboxylic acid of formula (XVII) under suitable conditions. Acids of formula (XVII) are commercially available.

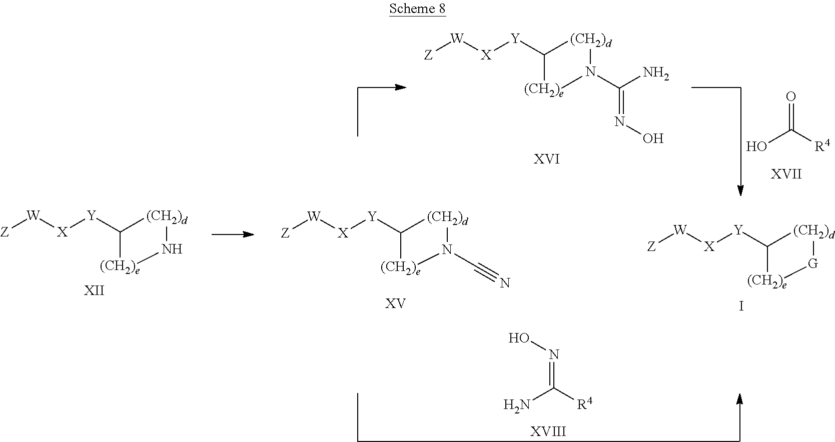

Other compounds of formula (I) may be prepared by methods analogous to those described above or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates, such as those defined above, may be of use in the synthesis of compounds of formula (I) and are therefore also included within the scope of the invention, including salts or protected derivatives thereof.

The processes for the production of compounds of formula (I) described above also represent further aspects of the invention.

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The compounds of the invention may offer advantages over compounds acting via different mechanisms for the treatment of the above mentioned disorders in that they may offer beta-cell protection, increased cAMP and insulin secretion and also slow gastric emptying.

The compounds of the invention may also be used for treating conditions characterised by low bone mass such asosteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine and loss of height.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I) may exhibit advantageous properties compared to known GPR119 agonists, for example, the compounds may exhibit improved potency or stability, or improved solubility thus improving absorption properties and bioavailability, or other advantageous properties for compounds to be used as pharmaceuticals.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, dipeptidyl peptidase IV inhibitors, GLP-1 agonists e.g. GLP-1 analogues and mimetics, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution containing 0.1% HCO₂H over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes.

Abbreviations and acronyms: Ac: Acetyl; Boc: tert-butoxycarbonyl; t-Bu: tent-Butyl; DCE: 1,2-dichloroethane; DCM: Dichloromethane; DEAD: Diethyl azodicarboxylate; DIAD: Diisopropyl azodicarboxylate; DIPEA: N,N-Diisopropylethylamine; DMF: Dimethylformamide; EDCI: 1-(3-Dimethylaminopropyl)-3-ethylcarbodiimide hydrochloride; Et: Ethyl; h: hour(s); min: minute/s; HOBt: 1-Hydroxybenzotriazole; IH: Isohexane; Me: Methyl; Ph: Phenyl; RT: Retention time; THF: Tetrahydrofuran.

The syntheses of the following compounds have been described elsewhere: (2R,3S)-2-Amino-3-hydroxybutane: U.S. Pat. No. 5,834,261; tert-Butyl 4-(3-hydroxypropyl)piperidine-1-carboxylate: Tetrahedron 1999, 55, 11619-11639; tert-Butyl 4-((E)-2-ethoxycarbonyl-1-methylvinyl)piperidine-1-carboxylate: U.S. Pat. No. 6,518,423. All other compounds were available from commercial sources.

### Preparation 1: 4-[3-(4-Methoxycarbonyl-3-methylphenoxy)propyl]piperidine-1-carboxylic acid tert-butyl ester

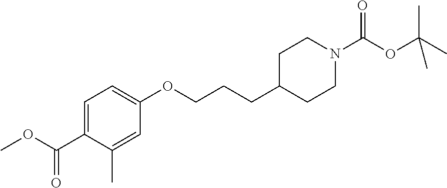

DIAD (8.00 mL, 40.9 mmol) was added to a stirred solution of 4-hydroxy-2-methyl-benzoic acid methyl ester (6.00 g, 37.4 mmol), tert-butyl 4-(3-hydroxypropyl)piperidine-1-carboxylate (8.25 g, 34.0 mmol) and PPh₃ (10.71 g, 40.9 mmol) in anhydrous THF (60 mL) at ambient temperature. After stirring for 7.5 h, the solvent was removed in vacuo, and the remainder was dissolved in EtOAc and washed with 2M NaOH (2×) and brine. The organic layer was dried (MgSO₄), concentrated under reduced pressure and the remainder was triturated with IH-Et₂O. The solid produced was filtered and washed with Et₂O. The combined washings and filtrate were concentrated under reduced pressure and purified by column chromatography (EtOAc—IH, 1:9) to afford the title compound: RT=4.48 min; m/z (ES⁺)=392.3[M+H]⁺.

### Preparation 2: 4-[3-(1-Cyanopiperidin-4-yl)propoxy]-2-methylbenzoic acid methyl ester

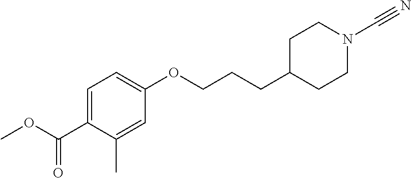

4M HCl in dioxane (7.7 mL) was added to a stirred solution of 4-[3-(4-methoxycarbonyl-3-methylphenoxy)propyl]piperidine-1-carboxylic acid tert-butyl ester (Preparation 1, 4.00 g, 10.2 mmol) in dioxane (10 mL) at ambient temperature. After 3 h, the mixture was diluted with Et₂O and the solid product formed was collected by filtration and washed with Et₂O to afford the hydrochloride salt of 2-methyl-4-(3-piperidin-4-ylpropoxy)-benzoic acid methyl ester: RT=2.65 min; m/z (ES⁺)=292.4 [M+H]⁺. To a stirred solution of this compound (10.77 g, 32.9 mmol) in DCM (140 mL) was added a slurry of NaHCO₃ (8.30 g, 98.7 mmol) in H₂O (100 mL) at 0° C. and the resulting mixture was treated with a solution of BrCN (4.18 g, 39.5 mmol) in DCM (22 mL). The reaction mixture was stirred at ambient temperature for 3 h, before being partitioned between H₂O and DCM. The organic phase was separated and dried (MgSO₄). Filtration and solvent evaporation provided the title compound: RT=3.87 min; m/z (ES⁺)=317.20 [M+H]⁺.

### Preparation 3: tert-Butyl 4-((E)-2-carboxy-1-methylvinyl)piperidine-1-carboxylate

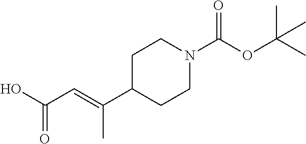

A solution of tert-butyl 4-((E)-2-ethoxycarbonyl-1-methylvinyl)piperidine-1-carboxylate (18.7 g, 62.9 mmol) in MeOH (90 mL) and H₂O (25 mL) was treated with 2M NaOH (94.5 mL, 189 mmol). The reaction was stirred for 16 h, the MeOH was removed under reduced pressure, then the remainder was partitioned between EtOAc and H₂O. The aqueous layer was separated and acidified to pH 2 with 12M HCl, before being extracted with EtOAc (2×). The organic extracts were washed with brine, dried (MgSO₄), filtered, and concentrated, then the remainder was recrystallised from EtOAc—IH to provide the title compound: m/z (ES⁻)=268.3[M−H]⁻.

### Preparation 4: tert-Butyl 4-((R)-2-carboxy-1-methylethyl)piperidine-1-carboxylate

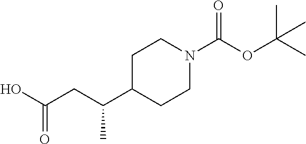

tert-Butyl 4-((E)-2-carboxy-1-methylvinyl)piperidine-1-carboxylate (Preparation 3, 130.0 g, 0.483 mol) was placed in a hydrogenation flask under an Ar atmosphere, then degassed MeOH (400 mL) was added. [Rh(norbornadiene)₂]BF₄ (1.80 g, 4.81 mmol) and (S)-1-[(R)-2-(di-tert-butylphosphino)ferrocenyl]ethylbis(2-methylphenyl)phosphine (2.90 g, 5.08 mmol) were placed in a separate Schlenk flask under Ar, before being treated with degassed MeOH (200 mL). This catalyst mixture was stirred for 15 min at ambient temperature, before being transferred via cannula into the hydrogenation flask. The Schlenk flask was rinsed with more degassed MeOH (100 mL). These washings were transferred to the hydrogenation flask, then more degassed MeOH (300 mL) was added. The hydrogenation flask was sealed, the Ar replaced by H₂, and the pressure set to 1.05 bar. The reaction mixture was heated to 35° C., and stirring/shaking was started. After 48 h, the reaction was stopped and a representative sample of the reaction mixture was analysed by HPLC and ¹H NMR. The conversion was 100% and the enantiomeric purity of the crude (R)-acid was 98.2%, as ascertained by the following HPLC method: Column: CHIRALPAK AD-H (previously used with CF₃CO₂H-containing solvents) 4.6×250 mm; Solvent: C₆H₁₄-iPrOH (97:3 isocratic); Temperature: 20° C.; Flow rate: 1 mL/min; UV-detection (210, 230 nm); Sample: 100 μL reaction solution dissolved with 1 mL MeOH. Retention times: (S)-acid: 19.3 min, (R)-acid: 20.6 min, starting enoic acid: 22.1 min. Isolation procedure: The MeOH was evaporated, then the crude hydrogenation product was dissolved in t-BuOMe and extracted with aqueous NaOH. The aqueous phase was added to a mixture of 1M HCl and EtOAc. The aqueous phase was extracted further with EtOAc, then the combined organic extracts were washed with brine and dried (MgSO₄). The title compound was isolated following filtration and complete removal of the solvent.

### Preparation 5: tert-Butyl 4-((R)-3-hydroxy-1-methylpropyl)piperidine-1-carboxylate

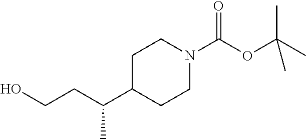

BH₃.THF (1M, 15.7 mL, 15.7 mmol) was added dropwise over 5 min to a stirred solution of tert-butyl 4-((R)-2-carboxy-1-methylethyl)piperidine-1-carboxylate (Preparation 4, 1.70 g, 6.30 mmol) in anhydrous THF at 0° C. After 1 h, the reaction was treated with Et₂O, then with 2M HCl. The organic layer was washed with brine, before being dried (Na₂SO₄). Filtration, solvent evaporation, and column chromatography (EtOAc—CH₂Cl₂, 1:3) provided the title compound: RT=3.17 min; m/z (ES⁺)=258.1 [M+H]⁺.

### Preparation 6: 4-[(R)-3-(4-Methanesulfonylphenoxy)-1-methylpropyl]piperidine-1-carboxylic acid tert-butyl ester

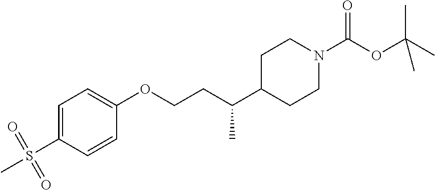

DEAD (10.8 mL, 68.4 mmol) was added to a stirred solution of tert-butyl 4-((R)-3-hydroxy-1-methylpropyl)piperidine-1-carboxylate (Preparation 5, 8.00 g, 31.1 mmol), 4-methanesulfonylphenol (5.63 g, 32.7 mmol) and PPh₃ (10.60 g, 40.4 mmol) in anhydrous THF (300 mL) at 0° C. After stirring at ambient temperature for 0.5 h, the solvent was removed in vacuo, and the remainder was dissolved in EtOAc to give a solution that was washed with 2M NaOH (2×) and brine. The organic layer was dried (MgSO₄), concentrated under reduced pressure and the remainder was triturated with IH-Et₂O. The solid produced was filtered and washed with Et₂O. The combined washings and filtrate were concentrated under reduced pressure and the residue was purified by column chromatography (EtOAc—IH, 3:7) to afford the title compound: RT=4.09 min; ink (ES⁺)=412.00 [M+H]⁺.

### Preparation 7: 4-[(R)-3-(4-Methanesulfonylphenoxy)-1-methylpropyl]piperidine-1-carbonitrile

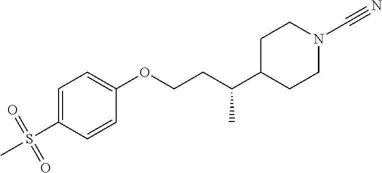

A mixture of 4-[(R)-3-(4-methanesulfonylphenoxy)-1-methylpropyl]piperidine-1-carboxylic acid tert-butyl ester (Preparation 6, 15.50 g, 37.7 mmol) and 4M HCl in dioxane (150 mL) was stirred at ambient temperature for 1 h. The solvent was removed in vacuo, azeotroping with toluene (2×), to afford the hydrochloride salt of 4-[(R)-3-(4-methanesulfonyl-phenoxy)-1-methylpropyl]piperidine: RT=2.19 min; m/z (ES⁺)=311.93 [M+H]⁺. To a stirred solution of this compound (2.50 g, 7.20 mmol) in DCM (200 mL) was added a slurry of NaHCO₃ (1.82 g, 21.7 mmol) in H₂O (100 mL) at 0° C. and the resulting mixture was treated with a solution of BrCN (917 mg, 8.70 mmol) in DCM (10 mL). The reaction mixture was stirred at 0° C. for 0.5 h and at ambient temperature for 1 h, before being partitioned between H₂O and DCM. The organic phase was separated, washed with water and brine, before being dried (MgSO₄). Filtration and solvent evaporation provided the title compound: RT=3.44 min; m/z (ES⁺)=336.97 [M+H]⁺.

### Preparation 8: N-Hydroxy-4-[(R)-3-(4-methanesulfonylphenoxy)-1-methylpropyl]piperidine-1-carboxamidine

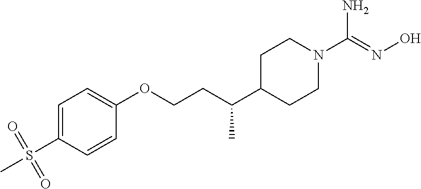

Hydroxylamine (50% aqueous solution, 146 μL, 4.80 mmol) was added to a stirred solution of 4-[(R)-3-(4-methanesulfonylphenoxy)-1-methylpropyl]piperidine-1-carbonitrile (Preparation 7, 400 mg, 1.19 mmol) in EtOH (6 mL) and the resulting mixture was stirred at 64° C. for 1 h. The reaction was concentrated, azeotroping with MeOH (2×), to afford the title compound: RT=2.38 min; m/z (ES⁺)=369.95 [M+H]⁺.

### Preparation 9: 1-(3-Chloromethyl-[1,2,4]oxadiazol-5-yl)-4-[(R)-3-(4-methanesulfonyl-phenoxy)-1-methylpropyl]piperidine

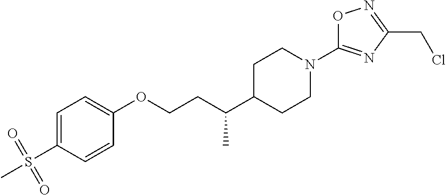

ZnCl₂ (1M in Et₂O, 3.94 mL, 3.94 mmol), followed by 2-chloro-N-hydroxyacetamidine (427 mg, 3.94 mmol) was added to a stirred solution of 4-[(R)-3-(4-methanesulfonylphenoxy)-1-methylpropyl]piperidine-1-carbonitrile (Preparation 7, 1.10 g, 3.28 mmol) in EtOAc (20 mL) and the resulting solution was stirred at ambient temperature for 16 h. The solvent was removed in vacuo, the remainder dissolved in EtOH (20 mL) and 12M HCl (2 mL) and the resulting solution stirred at 75° C. for 7.5 h. The EtOH was removed in vacuo and the remainder was adjusted to pH 7 with saturated aqueous NaHCO₃ solution. The mixture was extracted with EtOAc (2×), then the combined extracts were washed with brine and dried (MgSO₄). Filtration, solvent removal and purification by column chromatography (EtOAc—IH, 4:1) afforded the title compound: RT=3.76 min; m/z (ES⁺)=428.11 [M+H]⁺.

### Preparation 10: 4-((R)-3-Hydroxy-1-methylpropyl)piperidine-1-carbonitrile

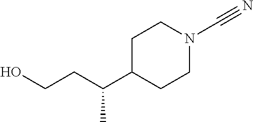

A mixture of tert-butyl 4-((R)-3-hydroxy-1-methylpropyl)piperidine-1-carboxylate (Preparation 5, 6.20 g, 14.9 mmol) and 4M HCl in dioxane (10 mL) were stirred at ambient temperature. After 3 h, the solvents were removed under reduced pressure to furnish the hydrochloride salt of (R)-3-piperidin-4-yl-butan-1-ol: δ_{H} ({CD₃}₂SO) 0.83 (d, 3H), 1.19-1.28 (m, 1H), 1.38-1.59 (m, 5H), 1.64-1.76 (m, 2H), 2.75-2.87 (m, 2H), 3.20-3.30 (m, 2H), 3.35-3.60 (m, 4H). A stirred mixture of this compound (930 mg, 4.8 mmol) and NaHCO₃ (1.61 g, 19.2 mmol) in DCM-H₂O (4:1, 15 mL) at 0° C. was treated with a solution of BrCN (610 mg, 5.8 mmol) in DCM (2 mL). The reaction was stirred at ambient temperature for 2 h, before being partitioned between H₂O and DCM. The organic phase was separated and dried (MgSO₄). Filtration, solvent evaporation, and column chromatography (EtOAc) provided the title compound: RT=2.45 min; m/z (ES⁺)=183.1 [M+H]⁺.

### Preparation 11: N-Hydroxy-4-((R)-3-hydroxy-1-methylpropyl)piperidine-1-carboxamidine

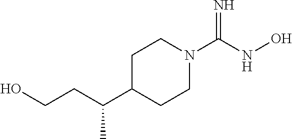

Hydroxylamine (50% aqueous solution, 1.88 mL, 28.5 mmol) was added to a solution of 4-((R)-3-hydroxy-1-methylpropyl)piperidine-1-carbonitrile (Preparation 10, 1.30 g, 7.14 mmol) in EtOH (15 mL) and the resulting solution heated at 60° C. for 45 min. The EtOH was removed in vacuo to afford the title compound: RT=1.65 min; m/z (ES⁺)=216.12 [M+H]⁺.

### Preparation 12: 2-{3-[4-((R)-3-Hydroxy-1-methylpropyl)piperidin-1-yl]-[1,2,4]oxadiazol-5-yl}pyrrolidine-1-carboxylic acid tert-butyl ester

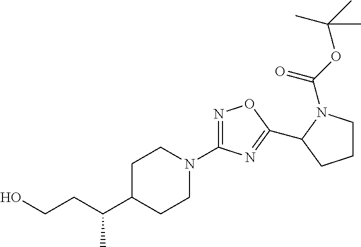

HOBt (980 mg, 7.25 mmol), EDCI (1.39 g, 6.04 mmol) and DIPEA (3.16 mL, 18.1 mmol) were added to a solution of N-hydroxy-4-((R)-3-hydroxy-1-methylpropyl)piperidine-1-carboxamidine (Preparation 11, 1.30 g, 6.04 mmol) and pyrrolidine-1,2-dicarboxylic acid 1-tert-butyl ester (1.30 g, 6.04 mmol) in DMF (7 mL) and the resulting solution stirred at ambient temperature for 72 h, followed by heating at 50° C. for 5 h. The DMF was removed in vacuo, then the residue was dissolved in H₂O and extracted with EtOAc (2×). The combined organic extracts were washed with saturated aqueous NaHCO₃ solution and brine, dried (MgSO₄), filtered and concentrated in vacuo. Purification by column chromatography (EtOAc—IH, 19:1) afforded the title compound: RT=3.60 min; m/z (ES⁺)=395.22 [M+H]⁺.

### Preparation 13: 2-(3-{4-[(R)-3-(4-Methanesulfonylphenoxy)-1-methylpropyl]piperidin-1-yl}-[1,2,4]oxadiazol-5-yl)pyrrolidine-1-carboxylic acid tert-butyl ester

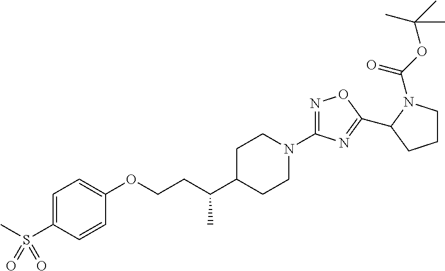

2-{3-[4-((R)-3-Hydroxy-1-methylpropyl)piperidin-1-yl]-[1,2,4]oxadiazol-5-yl}-pyrrolidine-1-carboxylic acid tert-butyl ester (Preparation 12, 500 mg, 1.27 mmol) and PPh₃ (501 mg, 1.91 mmol) were added to a solution of 4-methanesulfonylphenol (237 mg, 1.39 mmol) in THF (10 mL) followed by the dropwise addition of DIAD (375 μL, 1.91 mmol). The resulting reaction mixture was stirred at ambient temperature for 1 h and then concentrated in vacuo. The residue was dissolved in EtOAc, washed with 1M NaOH (2×50 mL) and brine, dried (MgSO₄), filtered and concentrated in vacuo. Purification by column chromatography (EtOAc—IH, 1:1) afforded the title compound: RT=4.06 min; m/z (ES⁺)=549.26 [M+H]⁺.

### Preparation 14: 4-[3-(4-Carboxy-3-methylphenoxy)propyl]piperidine-1-carboxylic acid tert-butyl ester

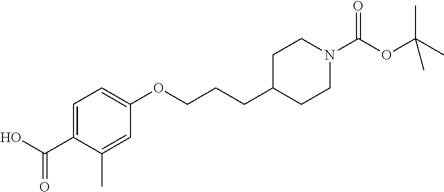

To a solution of 4-[3-(4-methoxycarbonyl-3-methylphenoxy)propyl]piperidine-1-carboxylic acid tert-butyl ester (Preparation 1, 6.00 g, 15.3 mmol) in MeOH (200 mL) and H₂O (20 mL) was added LiOH.H₂O (6.43 g, 153.3 mmol) and the resulting mixture was stirred at 40° C. for 16 h. The MeOH was evaporated off under reduced pressure, then the remainder was dissolved in H₂O (200 mL), washed with EtOAc and acidified to pH 4 with 2M HCl, before being extracted with EtOAc (2×). The combined organic extracts were washed with brine, dried (MgSO₄), filtered, and concentrated in vacuo to yield the title compound: RT=4.06 min; m/z (ES⁺)=378.22 [M+H]⁺.

### Preparation 15: 4-{3-[4-((S)-2,3-Dihydroxypropylcarbamoyl)-3-methylphenoxy]propyl}-piperidine-1-carboxylic acid tert-butyl ester

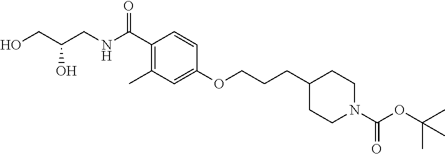

A solution of HOBt.H₂O (6.15 g, 45 mmol) and EDCI (8.71 g, 45 mmol) in CH₂Cl₂ (140 mL) was stirred at 20° C. for 15 min, before being treated with a solution of 4-[3-(4-carboxy-3-methylphenoxy)propyl]piperidine-1-carboxylic acid tert-butyl ester (Preparation 14, 13.70 g, 36 mmol) in CH₂Cl₂ (70 mL) over 30 min. After 16 h, a solution of (S)-3-amino-1,2-propanediol (4.14 g, 45 mmol) in MeOH (15 mL) was added slowly to the reaction mixture, followed by a solution of NEt₃ (2.94 g, 29 mmol) in CH₂Cl₂ (4 mL). The mixture was stirred further for 22 h, before being concentrated in vacuo. The residue was dissolved in CH₂Cl₂ (200 mL), then the solution was washed with 2M aqueous NaOH (3×70 mL) and 1M aqueous HCl (3×70 mL). The CH₂Cl₂ layer was separated and evaporated off under reduced pressure, then the residue was dissolved in EtOAc (200 mL). The EtOAc solution was washed with saturated aqueous NaHCO₃ (50 mL) and brine (50 mL), before being dried (MgSO₄). Filtration and solvent evaporation afforded the title compound: RT=3.37 min; m/z (ES⁺)=451.30 [M+H]⁺.

### Preparation 16: 4-[3-(1-Cyanopiperidin-4-yl)propoxy]-N—((S)-2,3-dihydroxypropyl)-2-methylbenzamide

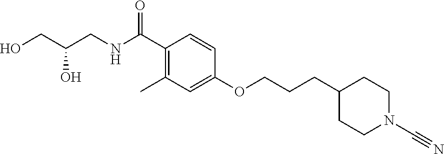

The tert-butoxycarbonyl group of 4-{3-[4-((S)-2,3-dihydroxypropylcarbamoyl)-3-methylphenoxy]propyl}piperidine-1-carboxylic acid tert-butyl ester (Preparation 15) was removed with HCl in dioxane, then the resultant amine was coupled with BrCN, employing procedures similar to those outlined in Preparation 10, to give the title compound: δ_{H} ({CD₃}₂SO) 1.15-1.25 (m, 2H), 1.35-1.50 (m, 3H), 1.68-1.80 (m, 5H), 2.36 (s, 3H), 2.95-3.05 (m, 2H), 3.12-3.21 (m, 1H), 3.30-3.40 (m, 4H), 3.59-3.63 (m, 1H), 3.94-4.01 (m, 2H), 4.56 (t, 1H), 4.77 (d, 1H), 6.76-6.81 (m, 2H), 7.35 (d, 1H), 7.97-8.00 (m, 1H).

### Preparation 17: N—((S)-2,3-Dihydroxypropyl)-4-{3-[1-(N-hydroxycarbamimidoyl)piperidin-4-yl]propoxy}-2-methylbenz amide

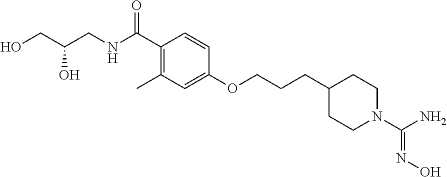

Reaction of 4-[3-(1-cyanopiperidin-4-yl)propoxy]-N—((S)-2,3-dihydroxypropyl)-2-methylbenzamide (Preparation 16) with hydroxylamine, utilising a procedure similar to that delineated in Preparation 11, furnished the title compound: RT=2.18 min; m/z (ES⁺)=409.20 [M+H]⁺.

### Preparation 18: 4-{3-[4-((R)-2-Hydroxy-1-methylethylcarbamoyl)-3-methylphenoxy]propyl}-piperidine-1-carboxylic acid tert-butyl ester

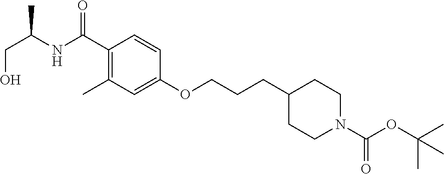

Condensation of 4-[3-(4-carboxy-3-methylphenoxy)propyl]piperidine-1-carboxylic acid tert-butyl ester (Preparation 14) with (R)-2-aminopropan-1-ol, employing a procedure similar to that delineated for Preparation 15, furnished the title compound: RT=3.60 min; m/z (ES⁺)=435.27 [M+H]⁺.

### Preparation 19: 4-[3-(1-Cyanopiperidin-4-yl)propoxy]-N—((R)-2-hydroxy-1-methylethyl)-2-methylbenzamide

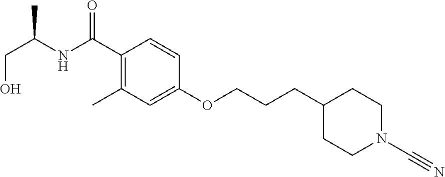

The tert-butoxycarbonyl group of 4-{3-[4-((R)-2-hydroxy-1-methylethylcarbamoyl)-3-methylphenoxy]propyl}piperidine-1-carboxylic acid tert-butyl ester (Preparation 18) was removed with HCl in dioxane, then the resultant amine was coupled with BrCN, employing procedures similar to those outlined in Preparation 10, to give the title compound: RT=2.97 min; m/z (ES⁺)=360.22 [M+H]⁺.

### Preparation 20: 4-{3-[1-(N-Hydroxycarbamimidoyl)piperidin-4-yl]propoxy}-N—((R)-2-hydroxy-1-methylethyl)-2-methylbenzamide

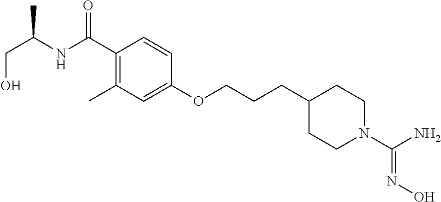

Reaction of 4-[3-(1-cyanopiperidin-4-yl)propoxy]-N—((R)-2-hydroxy-1-methylethyl)-2-methylbenzamide (Preparation 19) with hydroxylamine, utilising a procedure similar to that delineated in Preparation 11, furnished the title compound: RT=2.17 min; m/z (ES⁺)=393.20 [M+H]⁺.

### Preparation 21: 4-{3-[1-(N-Hydroxycarbamimidoyl)piperidin-4-yl]propoxy}-2-methylbenzoic acid methyl ester

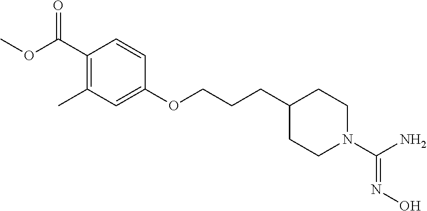

A mixture of aqueous NH₂OH solution (50 wt %, 3.7 mL) and EtOH (50 mL) was added over a period of 2 h to a stirred solution of 4-[3-(1-cyanopiperidin-4-yl)propoxy]-2-methylbenzoic acid methyl ester (Preparation 2, 9.48 g, 30.0 mmol) in EtOH (50 mL). After stirring at ambient temperature for 18 h, the solvent was removed and the residue was further dried through repeated concentration from PhMe to furnish the title compound: RT=2.59 min; m/z (ES⁺)=350.18 [M+H]⁺.

### Preparation 22: 2,2-Difluoro-N-hydroxyacetamidine

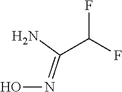

A stirred solution of difluoroacetonitrile (2.21 g, 28.7 mmol) in EtOH (5 mL) was treated carefully with a 50 wt % solution of hydroxylamine in H₂O (2.08 g, 31.6 mmol). The mixture was stirred further for 16 h, before being concentrated under reduced pressure. The residue was dried through repeated concentration from PhMe, then the oil that remained was partitioned between EtOAc and H₂O. The aqueous phase was extracted further with EtOAc (2×), then the combined organic extracts were dried (Na₂SO₄), filtered, and concentrated to furnish the title compound: m/z (ES⁺)=111.02 [M+H]⁺.

### Preparation 23: 2,2-Difluoro-N-hydroxypropionamidine

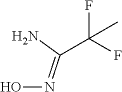

2,2-Difluoropropionitrile was reacted with hydroxylamine, employing a procedure similar to that outlined in Preparation 22, to afford the title compound: m/z (ES⁺)=125.03 [M+H]⁺.

### Example 1

### 4-{3-[1-(3-Cyclopropyl-[1,2,4]oxadiazol-5-yl)piperidin-4-yl]propoxy}-2-methylbenzoic acid methyl ester

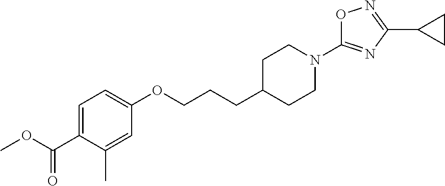

ZnCl₂ (1M in Et₂O, 34.3 mL, 34.3 mmol) was slowly added to a stirred solution of 4-[3-(1-cyanopiperidin-4-yl)propoxy]-2-methylbenzoic acid methyl ester (Preparation 2, 9.06 g, 28.6 mmol) and N-hydroxycyclopropanecarboxamidine (3.47 g, 34.3 mmol) in EtOAc (145 mL) and the resulting solution was stirred at 60° C. for 16 h. The reaction was cooled to ambient temperature and the white precipitate that had formed was collected and washed with EtOAc. This precipitate was dissolved in MeOH (135 mL) and 12M HCl (13.5 mL), then the solution was stirred at 65° C. for 5 h. The MeOH was removed in vacuo, and the remainder was adjusted to pH 7 with saturated aqueous NaHCO₃ solution. The mixture was extracted with EtOAc (3×), then the combined extracts were washed with brine and dried (MgSO₄). Filtration, solvent removal and purification by column chromatography (IH-EtOAc, 3:1) afforded the title compound: RT=4.27 min; m/z (ES⁺)=400.23 [M+H]⁺.

### Example 2

### 4-{3-[1-(3-Cyclobutyl-[1,2,4]oxadiazol-5-yl)piperidin-4-yl]propoxy}-2-methyl-benzoic acid methyl ester

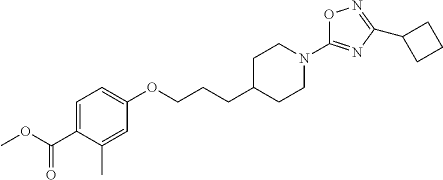

ZnCl₂ (1M in Et₂O, 6.65 mL, 6.65 mmol) was slowly added to a stirred solution of 4-[3-(1-cyanopiperidin-4-yl)propoxy]-2-methylbenzoic acid methyl ester (Preparation 2, 1.00 g, 3.16 mmol) and N-hydroxycyclobutanecarboxamidine (760 mg, 6.65 mmol) in EtOAc (50 mL) and the resulting solution was stirred at 35° C. for 16 h. The reaction was cooled to ambient temperature and the white precipitate that had formed was collected and washed with Et₂O. This precipitate was dissolved in MeOH (50 mL) and 12M HCl (6 mL), then the solution was stirred at 60° C. for 16 h. The MeOH was removed in vacuo, and the remainder was adjusted to pH 7 with saturated aqueous NaHCO₃ solution. The mixture was extracted with DCM (3×), then the combined extracts were dried (MgSO₄). Filtration and solvent removal afforded the title compound: RT=4.32 min; m/z (ES⁺)=414.19 [M+H]⁺.

### Example 3

### 4-{3-[1-(3-Methoxymethyl-[1,2,4]oxadiazol-5-yl)piperidin-4-yl]propoxy}-2-methylbenzoic acid methyl ester

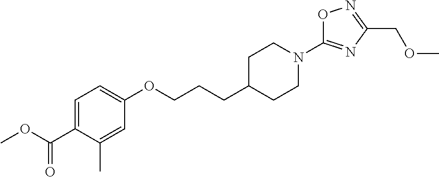

The title compound was synthesized from 4-[3-(1-cyanopiperidin-4-yl)propoxy]-2-methylbenzoic acid methyl ester (Preparation 2) and N-hydroxy-2-methoxyacetamidine employing a procedure similar to that outlined in Example 2: RT=3.98 min; m/z (ES⁺)=404.20 [M+H]⁺.

### Example 4

### 4-(3-{1-[5-(1-Fluoro-1-methylethyl)-[1,2,4]oxadiazol-3-yl]piperidin-4-yl}-propoxy)-2-methylbenzoic acid methyl ester

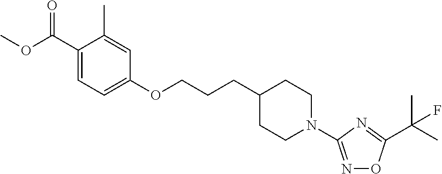

To a solution of 4-{3-[1-(N-hydroxycarbamimidoyl)piperidin-4-yl]propoxy}-2-methylbenzoic acid methyl ester (Preparation 21, 2.44 g, 7.0 mmol) and 2-fluoroisobutyric acid (742 mg, 7.0 mmol) in DMF (20 mL) was added HOBt (107 mg, 0.70 mmol), EDCI (1.74 g, 9.1 mmol) and NEt₃ (2.2 mL, 16.1 mmol). After stirring at ambient temperature for 18 h the solvent was removed in vacuo and the residue was redissolved in EtOAc (400 mL). The EtOAc solution was washed with 1M HCl solution, 1M NaOH solution and brine, before being dried (MgSO₄), filtered and concentrated to give a residue which was purified by column chromatography (IH-EtOAc, 3:1) to afford the title compound: RT=4.84 min; m/z (ES⁺)=420.19 [M+H]⁺.

### Example 5

### 4-(3-{1-[5-(1,1-Difluoroethyl)-[1,2,4]oxadiazol-3-yl]piperidin-4-yl}propoxy)-2-methylbenzoic acid methyl ester

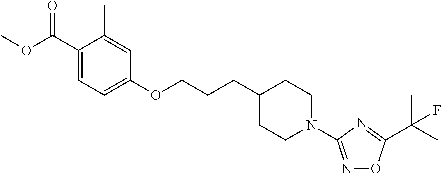

4-{3-[1-(N-Hydroxycarbamimidoyl)piperidin-4-yl]propoxy}-2-methylbenzoic acid methyl ester (Preparation 21) was reacted with 2,2-difluoropropionic acid, utilizing a procedure similar to that outlined in Example 4, to furnish the title compound: RT=4.52 min; m/z (ES⁺)=424.23 [M+H]⁺.

### Example 6

### 4-{3-[1-(3-Difluoromethyl-[1,2,4]oxadiazol-5-yl)piperidin-4-yl]propoxy}-2-methylbenzoic acid methyl ester

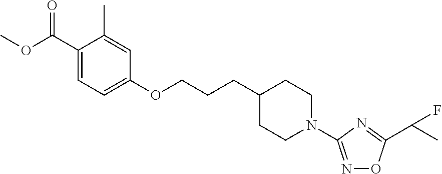

The title compound was synthesized from 4-[3-(1-cyanopiperidin-4-yl)propoxy]-2-methylbenzoic acid methyl ester (Preparation 2) and 2,2-difluoro-N-hydroxyacetamidine (Preparation 22) employing a procedure similar to that outlined in Example 2: RT=4.50 min; m/z (ES⁺)=410.16 [M+H]⁺.

### Example 7

### 4-(3-{1-[3-(1,1-Difluoroethyl)-[1,2,4]oxadiazol-5-yl]piperidin-4-yl}propoxy)-2-methylbenzoic acid methyl ester

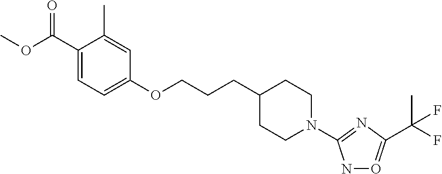

The title compound was synthesized from 4-[3-(1-cyanopiperidin-4-yl)propoxy]-2-methylbenzoic acid methyl ester (Preparation 2) and 2,2-difluoro-N-hydroxypropionamidine (Preparation 23) employing a procedure similar to that outlined in Example 2: RT=4.59 min; m/z (ES⁺)=424.21 [M+H]⁺.

### Example 8

### 4-{3-[1-(3-Cyclopropyl-[1,2,4]oxadiazol-5-yl)piperidin-4-yl]propoxy}-2-methyl-benzoic acid

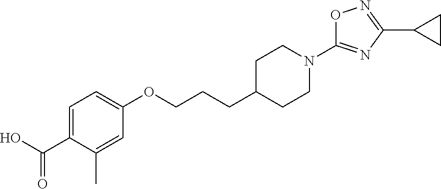

A mixture of LiOH.H₂O (7.92 g, 189 mmol) and 4-{3-[1-(3-cyclopropyl-[1,2,4]oxadiazol-5-yl)piperidin-4-yl]propoxy}-2-methylbenzoic acid methyl ester (Example 1, 7.41 g, 18.9 mmol) in MeOH (220 mL) and H₂O (22 mL) was heated at 50° C. for 38 h. The MeOH was removed under reduced pressure, then the remainder was partitioned between 2M NaOH and EtOAc. The aqueous phase was acidified to pH 1 with 2M HCl, before being extracted with EtOAc (3×). The combined organic extracts were washed with brine, dried (MgSO₄), filtered and concentrated in vacuo to afford the title compound: RT=3.86 min; m/z (ES⁺)=386.22 [M+H]⁺.

### Example 9

### 4-{3-[1-(3-Cyclopropyl-[1,2,4]oxadiazol-5-yl)piperidin-4-yl]propoxy}-N-(2-hydroxyethyl)-2-methylbenzamide

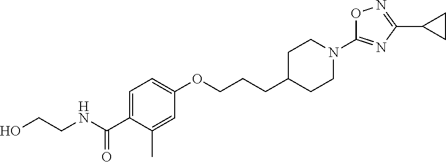

HOBt.H₂O (822 mg, 6.08 mmol) was added to a stirred solution of 4-{3-[1-(3-cyclopropyl-[1,2,4]oxadiazol-5-yl)piperidin-4-yl]propoxy}-2-methylbenzoic acid (Example 8, 1.75 g, 4.60 mmol) and EDCI (1.17 g, 6.08 mmol) in THF (110 mL). After 15 min, 2-amino-ethanol (570 mg, 9.35 mmol) was added and the resulting mixture was heated at 40° C. for 16 h. The THF was removed in vacuo and the residue was partitioned between EtOAc and 2M NaOH. The organic phase was separated and washed with 2M NaOH, 1M HCl and brine, before being dried (MgSO₄). Filtration, solvent evaporation, and purification by column chromatography (EtOAc-MeOH, 97:3) afforded the title compound: δ_{H} (CDCl₃) 0.93-1.03 (m, 4H), 1.22-1.36 (m, 2H), 1.43-1.52 (m, 2H), 1.53-1.60 (m, 1H), 1.78-1.94 (m, 5H), 2.47-2.57 (m, 4H), 2.99-3.10 (m, 2H), 3.61-3.67 (m, 2H), 3.84-3.90 (m, 2H), 4.0 (t, 2H), 4.09-4.17 (m, 2H), 6.15-6.24 (m, 1H), 6.70-6.75 (m, 1H), 6.75-6.79 (m, 1H), 7.40 (d, 1H); RT=3.38 min; m/z (ES⁺)=429.31 [M+H]⁺.

The amides listed in Table 1 were synthesised by condensing 4-{3-[1-(3-cyclopropyl-[1,2,4]oxadiazol-5-yl)piperidin-4-yl]propoxy}-2-methylbenzoic acid (Example 8) with the appropriate amine, employing a procedure similar to that outlined in Example 9.

TABLE 1

| Ex | Structure | Name | Spectra: LCMS |
|---|---|---|---|
| 10 | 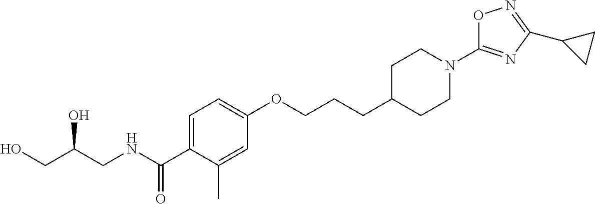 | 4-{3-[1-(3- Cyclopropyl- [1,2,4]oxadiazol-5-yl)- piperidin-4- yl]propoxy}-N- ((S)-2,3- dihydroxypropyl)-2- methylbenzamide | RT = 3.23 min; m/z (ES⁺) = 459.26 [M + H]⁺ |
| 11 | 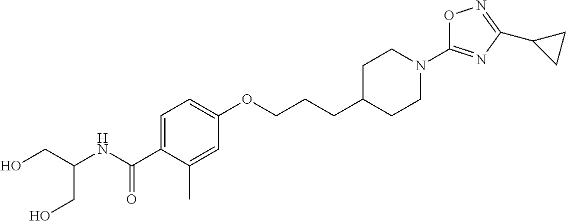 | 4-{3-[1-(3- Cyclopropyl- [1,2,4]oxadiazol-5-yl)- piperidin-4-yl] propoxy}-N-(2- hydroxy-1- hydroxymethyl- ethyl)-2- methylbenzamide | RT = 3.25 min; m/z (ES⁺) = 459.27 [M + H]⁺ |
| 12 | 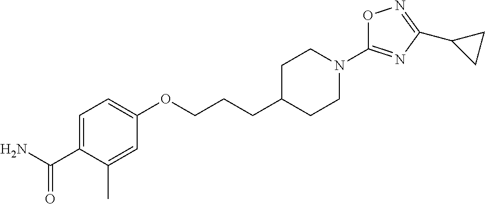 | 4-{3-[1-(3- Cyclopropyl- [1,2,4]oxadiazol-5-yl)- piperidin-4-yl] propoxy}-2- methylbenzamide | RT = 3.48 min; m/z (ES⁺) = 385.25 [M + H]⁺ |
| 13 | 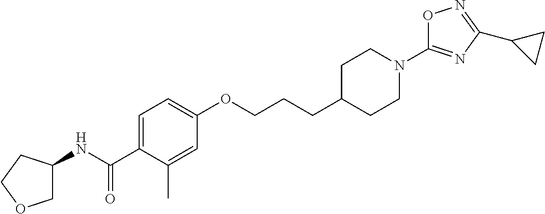 | 4-{3-[1-(3- Cyclopropyl- [1,2,4]oxadiazol-5-yl)- piperidin-4-yl] propoxy}-2- methyl-N-(R)- tetrahydrofuran- 3-ylbenzamide | RT = 3.57 min; m/z (ES⁺) = 455.26 [M + H]⁺ |

### Example 14

### 4-{3-[1-(3-Cyclobutyl-[1,2,4]oxadiazol-5-yl)piperidin-4-yl]propoxy}-2-methyl-benzoic acid

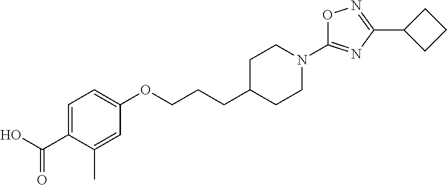

The title compound was synthesized from 4-{3-[1-(3-cyclopropyl-[1,2,4]oxadiazol-5-yl)piperidin-4-yl]propoxy}-2-methylbenzoic acid methyl ester (Example 2, 1.27 g, 3.07 mmol) employing a procedure similar to that outlined in Example 8: RT=3.88 min; m/z (ES⁺)=400.17 [M+H]⁺.

### Example 15

### 4-{3-[1-(3-Cyclobutyl-[1,2,4]oxadiazol-5-yl)piperidin-4-yl]propoxy}-N-(2-hydroxy-1-hydroxymethylethyl)-2-methylbenzamide

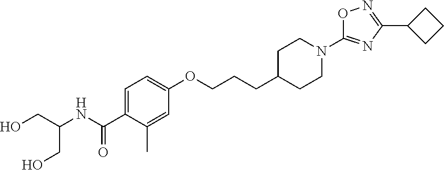

The title compound was synthesized from 4-{3-[1-(3-cyclobutyl-[1,2,4]oxadiazol-5-yl)-piperidin-4-yl]propoxy}-2-methylbenzoic acid (Example 14, 100 mg, 249 μmol) and 2-amino-propane-1,3-diol (34.0 mg, 374 μmol) employing a procedure similar to that outlined in Example 9: RT=3.24 min; m/z (ES⁺)=473.14 [M+H]⁺.

### Example 16

### 4-{3-[1-(3-Cyclobutyl-[1,2,4]oxadiazol-5-yl)piperidin-4-yl]propoxy}-2-methyl-benzamide

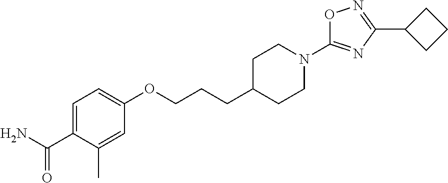

The title compound was synthesized from 4-{3-[1-(3-cyclobutyl-[1,2,4]oxadiazol-5-yl)-piperidin-4-yl]propoxy}-2-methylbenzoic acid (Example 14, 100 mg, 249 μmol) and ammonium chloride (20.0 mg, 374 μmol) employing a procedure similar to that outlined in Example 9: RT=3.54 min; m/z (ES⁺)=399.17 [M+H]⁺.

### Example 17

### 4-{3-[1-(3-Methoxymethyl-[1,2,4]oxadiazol-5-yl)piperidin-4-yl]propoxy}-2-methylbenzoic acid

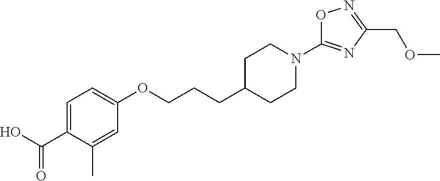

The title compound was synthesized from 4-{3-[1-(3-methoxymethyl-[1,2,4]oxadiazol-5-yl)piperidin-4-yl]propoxy}-2-methylbenzoic acid methyl ester (Example 3) employing a procedure similar to that outlined in Example 8: RT=3.47 min; m/z (ES⁺)=390.15 [M+H]⁺.

### Example 18

### N-(2-Hydroxy-1-hydroxymethylethyl)-4-{3-[1-(3-methoxymethyl-[1,2,4]oxadiazol-5-yl)piperidin-4-yl]propoxy}-2-methylbenzamide

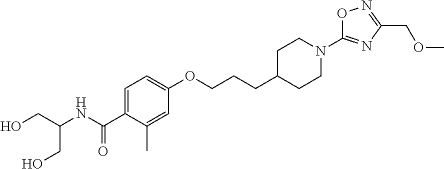

The title compound was synthesized from 4-{3-[1-(3-methoxymethyl-[1,2,4]oxadiazol-5-yl)piperidin-4-yl]propoxy}-2-methylbenzoic acid (Example 17, 100 mg, 257 μmol) and 2-aminopropane-1,3-diol (35.0 mg, 386 μmol) employing a procedure similar to that outlined in Example 9: RT=2.97 min; m/z (ES⁺)=463.21 [M+H]⁺.

### Example 19

### 4-{3-[1-(3-Methoxymethyl-[1,2,4]oxadiazol-5-yl)piperidin-4-yl]propoxy}-2-methylbenzamide

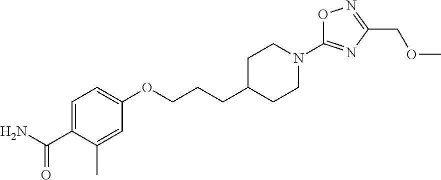

The title compound was synthesized from 4-{3-[1-(3-methoxymethyl-[1,2,4]oxadiazol-5-yl)piperidin-4-yl]propoxy}-2-methylbenzoic acid (Example 17, 50.0 mg, 125 μmol) and ammonium chloride (20.0 mg, 374 μmol) employing a procedure similar to that outlined in Example 9: RT=3.20 min; m/z (ES⁺)=389.16 [M+H]⁺.

### Example 20

### N—((R)-2-Hydroxy-1-methylethyl)-4-{3-[1-(3-methoxymethyl-[1,2,4]oxadiazol-5-yl)piperidin-4-ylpropoxy}-2-methylbenzamide

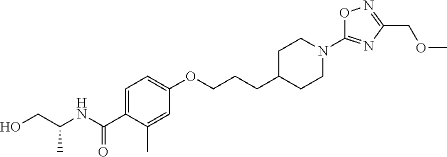

The title compound was synthesized from 4-{3-[1-(3-methoxymethyl-[1,2,4]oxadiazol-5-yl)piperidin-4-yl]propoxy}-2-methylbenzoic acid (Example 17, 100 mg, 257 μmol) and (R)-2-aminopropan-1-ol (35.0 mg, 386 μmol) employing a procedure similar to that outlined in Example 9: RT=3.17 min; m/z (ES⁺)=447.21 [M+H]⁺.

### Example 21

### 4-(3-{1-[5-(1-Fluoro-1-methylethyl)-[1,2,4]oxadiazol-3-yl]piperidin-4-yl}-propoxy)-2-methylbenzoic acid

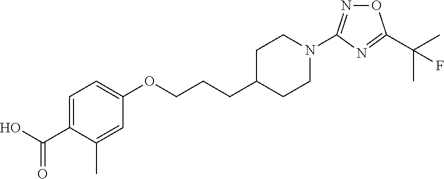

To a solution of 4-(3-{1-[5-(1-fluoro-1-methylethyl)-[1,2,4]oxadiazol-3-yl]piperidin-4-yl}propoxy)-2-methylbenzoic acid methyl ester (Example 4, 1.59 g, 3.79 mmol) in MeOH (100 mL) and H₂O (20 mL) was added LiOH.H₂O (1.61 g, 38.4 mmol) and the mixture was stirred at 50° C. for 12 h. Most of the MeOH was removed in vacuo, then more H₂O (100 mL) was added, and the mixture was acidified to pH 3 with 1M HCl solution. The precipitate was extracted into EtOAc, then the combined EtOAc extracts were washed with brine and dried (MgSO₄). Filtration and removal of the solvent afforded the title compound: RT=4.22 min; m/z (ES⁺)=406.20 [M+H]⁺.

### Example 22

### 4-(3-{1-[5-(1,1-Difluoroethyl)-[1,2,4]oxadiazol-3-yl]piperidin-4-yl}propoxy)-2-methylbenzoic acid

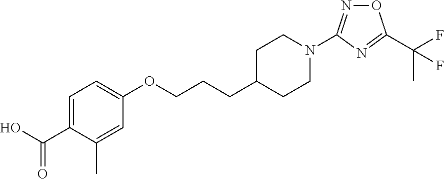

Saponification of 4-(3-{1-[5-(1,1-difluoroethyl)-[1,2,4]oxadiazol-3-yl]piperidin-4-yl}-propoxy)-2-methylbenzoic acid methyl ester (Example 5), using a method similar to that outlined in Example 21, afforded the title compound: RT=4.25 min; m/z (ES⁺)=410.18 [M+H]⁺.

### Example 23

### 4-{3-[1-(3-Difluoromethyl-[1,2,4]oxadiazol-5-yl)piperidin-4-yl]propoxy}-2-methylbenzoic acid

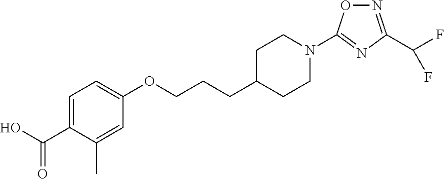

Saponification of 4-{3-[1-(3-difluoromethyl-[1,2,4]oxadiazol-5-yl)piperidin-4-yl]-propoxy}-2-methylbenzoic acid methyl ester (Example 6), using a method similar to that outlined in Example 21, furnished the title compound: RT=3.95 min; m/z (ES⁺)=396.14 [M+H]⁺.

### Example 24

### 4-(3-{1-[3-(1,1-Difluoroethyl)-[1,2,4]oxadiazol-5-yl]piperidin-4-yl}propoxy)-2-methylbenzoic acid

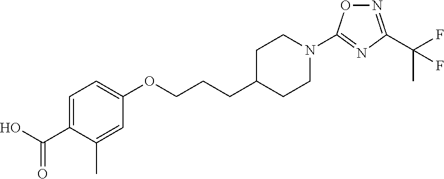

Saponification of 4-(3-{1-[3-(1,1-difluoroethyl)-[1,2,4]oxadiazol-5-yl]piperidin-4-yl}-propoxy)-2-methylbenzoic acid methyl ester (Example 7), using a method similar to that outlined in Example 21, furnished the title compound: RT=4.03 min; m/z (ES⁺)=410.19 [M+H]⁺.

The amides listed in Table 2 were synthesised by condensing the appropriate acid with the appropriate amine, employing a procedure similar to that outlined in Example 9.

TABLE 2

| Ex | Structure | Name | Spectra: LCMS |
|---|---|---|---|
| 25 | 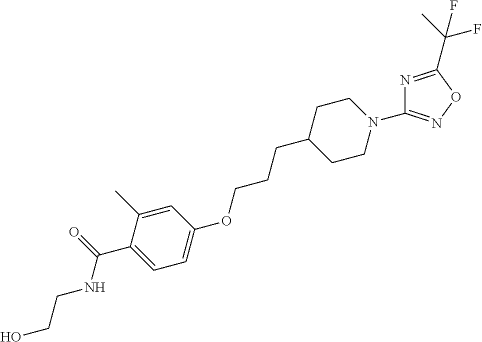 | 4-(3-{1-[5-(1,1-Difluoro- ethyl)-[1,2,4]oxadiazol-3-yl]- piperidin-4-yl}propoxy)-N-(2- hydroxyethyl)-2-methyl- benzamide | RT = 3.65 min; m/z (ES⁺) = 453.23 [M + H]⁺ |
| 26 | 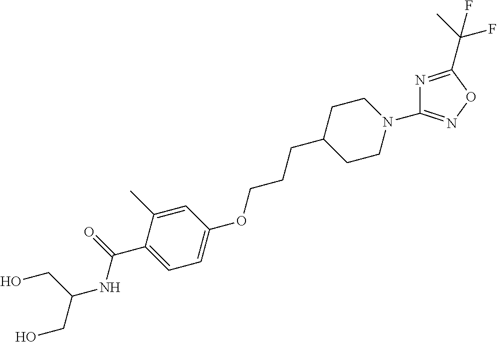 | 4-(3-{1-[5-(1,1-Difluoro- ethyl)-[1,2,4]oxadiazol-3-yl]- piperidin-4-yl}propoxy)-N-(2- hydroxy-1-hydroxymethyl- ethyl)-2-methylbenzamide | RT = 3.55 min; m/z (ES⁺) = 483.25 [M + H]⁺ |
| 27 | 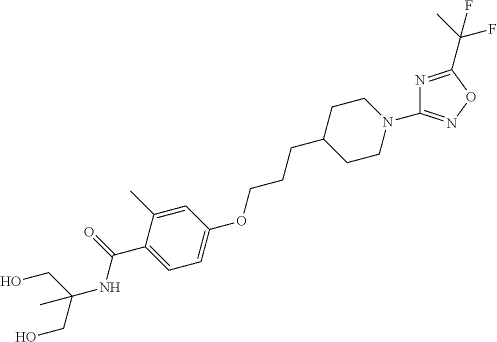 | 4-(3-{1-[5-(1,1-Difluoro- ethyl)-[1,2,4]oxadiazol-3-yl]- piperidin-4-yl}propoxy)-N-(2- hydroxy-1-hydroxymethyl-1- methylethyl)-2-methyl- benzamide | RT = 3.72 min; m/z (ES⁺) = 497.26 [M + H]⁺ |
| 28 | 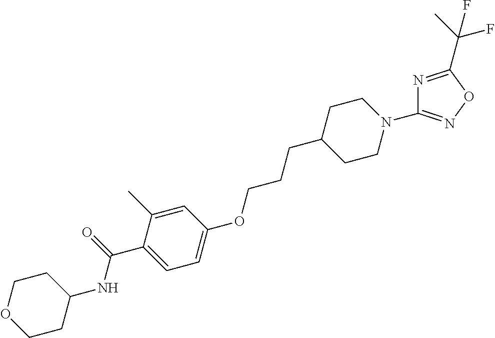 | 4-(3-{1-[5-(1,1-Difluoro- ethyl)-[1,2,4]oxadiazol-3-yl]- piperidin-4-yl}propoxy)-2- methyl-N-(tetrahydropyran-4- yl)benzamide | RT = 4.00 min; m/z (ES⁺) = 493.27 [M + H]⁺ |
| 29 | 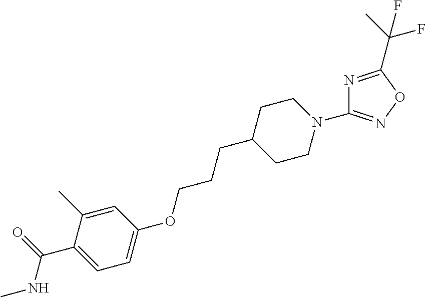 | 4-(3-{1-[5-(1,1-Difluoro- ethyl)-[1,2,4]oxadiazol-3-yl]- piperidin-4-yl}propoxy)-2,N- dimethylbenzamide | RT = 3.97 min; m/z (ES⁺) = 423.23 [M + H]⁺ |
| 30 | 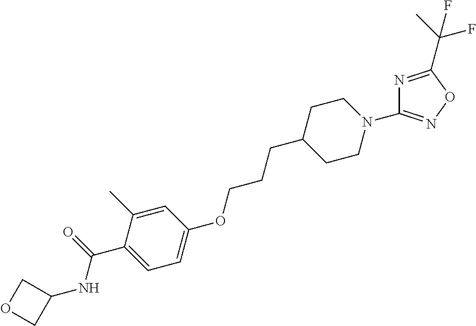 | 4-(3-{1-[5-(1,1-Difluoro- ethyl)-[1,2,4]oxadiazol-3-yl]- piperidin-4-yl}propoxy)-2- methyl-N-oxetan-3-yl- benzamide | RT = 3.88 min; m/z (ES⁺) = 465.23 [M + H]⁺ |
| 31 | 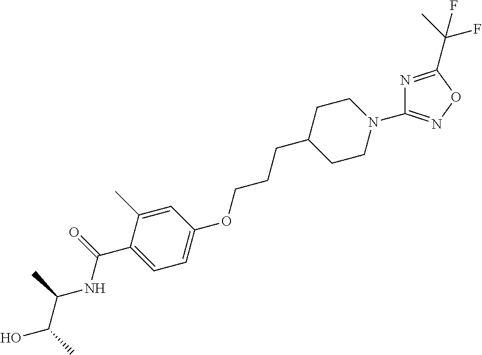 | 4-(3-{1-[5-(1,1-Difluoro- ethyl)-[1,2,4]oxadiazol-3-yl]- piperidin-4-yl}propoxy)-N- ((1R,2S)-2-hydroxy-1-methyl- propyl)-2-methylbenzamide | RT = 3.93 min; m/z (ES⁺) = 481.27 [M + H]⁺ |
| 32 | 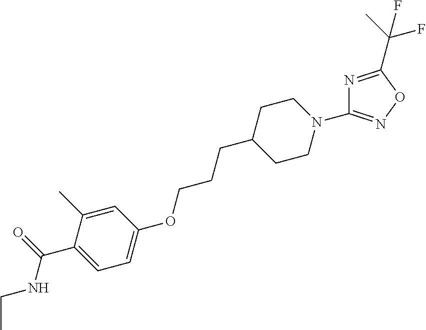 | 4-(3-{1-[5-(1,1-Difluoro- ethyl)-[1,2,4]oxadiazol-3-yl]- piperidin-4-yl}propoxy)-N- ethyl-2-methylbenzamide | RT = 4.12 min; m/z (ES⁺) = 437.24 [M + H]⁺ |
| 33 | 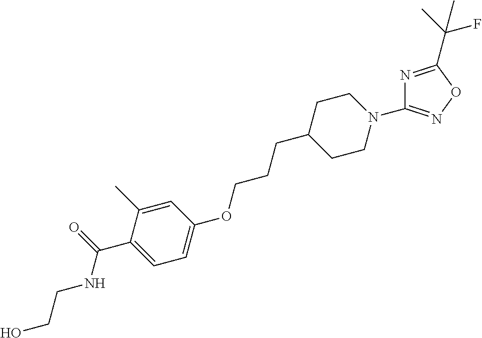 | 4-(3-{1-[5-(1-Fluoro-1-methyl- ethyl)-[1,2,4]oxadiazol-3-yl]- piperidin-4-yl}propoxy)-N-(2- hydroxyethyl)-2-methyl- benzamide | RT = 3.63 min; m/z (ES⁺) = 449.26 [M + H]⁺ |
| 34 | 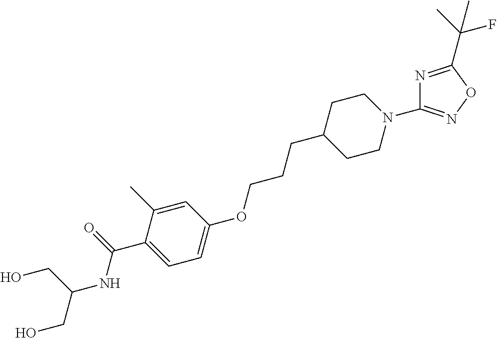 | 4-(3-{1-[5-(1-Fluoro-1-methyl- ethyl)-[1,2,4]oxadiazol-3-yl]- piperidin-4-yl}propoxy)-N-(2- hydroxy-1-hydroxymethyl- ethyl)-2-methylbenzamide | RT = 3.42 min; m/z (ES⁺) = 479.27 [M + H]⁺ |
| 35 | 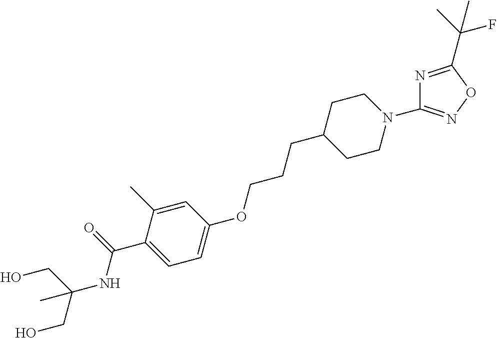 | 4-(3-{1-[5-(1-Fluoro-1-methyl- ethyl)-[1,2,4]oxadiazol-3-yl]- piperidin-4-yl}propoxy)-N-(2- hydroxy-1-hydroxymethyl-1- methylethyl)-2-methyl- benzamide | RT = 3.68 min; m/z (ES⁺) = 493.28 [M + H]⁺ |
| 36 | 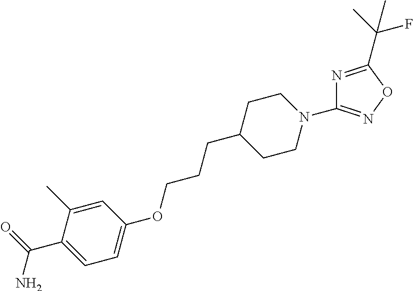 | 4-(3-{1-[5-(1-Fluoro-1-methyl- ethyl)-[1,2,4]oxadiazol-3-yl]- piperidin-4-yl}propoxy)-2- methylbenzamide | RT = 3.75 min; m/z (ES⁺) = 405.24 [M + H]⁺ |
| 37 | 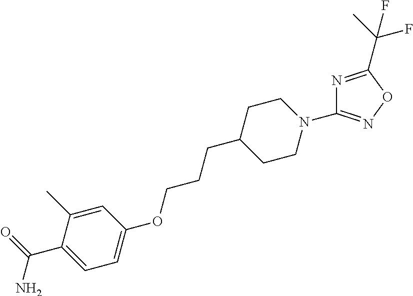 | 4-(3-{1-[5-(1,1-Difluoro- ethyl)-[1,2,4]oxadiazol-3-yl]- piperidin-4-yl}propoxy)-2- methylbenzamide | RT = 3.84 min; m/z (ES⁺) = 409.21 [M + H]⁺ |
| 38 | 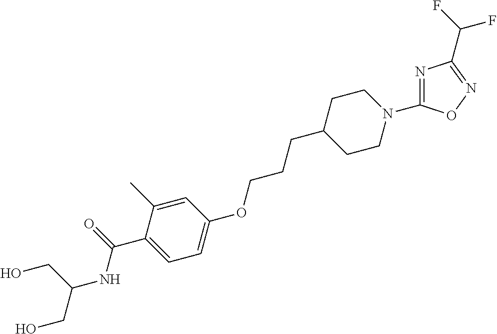 | 4-{3-[1-(3-Difluoromethyl- [1,2,4]oxadiazol-5-yl)- piperidin-4-yl]propoxy}-N-(2- hydroxy-1-hydroxymethyl- ethyl)-2-methylbenzamide | RT = 3.27 min; m/z (ES⁺) = 469.19 [M + H]⁺ |
| 39 | 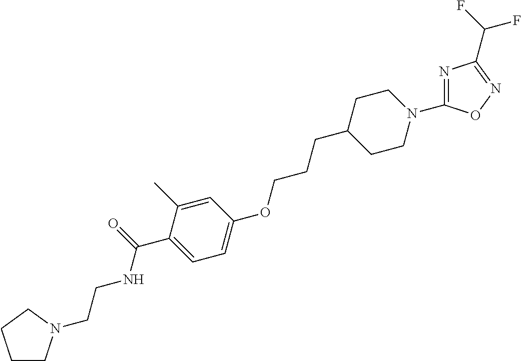 | 4-{3-[1-(3-Difluoromethyl- [1,2,4]oxadiazol-5-yl)- piperidin-4-yl]propoxy}-2- methyl-N-(2-pyrrolidin-1-yl- ethyl)benzamide | RT = 2.72 min; m/z (ES⁺) = 492.25 [M + H]⁺ |
| 40 | 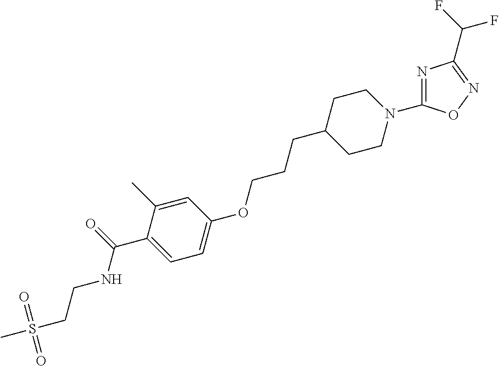 | 4-{3-[1-(3-Difluoromethyl- [1,2,4]oxadiazol-5-yl)- piperidin-4-yl]propoxy}-N-(2- methanesulfonylethyl)-2- methylbenzamide | RT = 3.55 min; m/z (ES⁺) = 501.21 [M + H]⁺ |
| 41 | 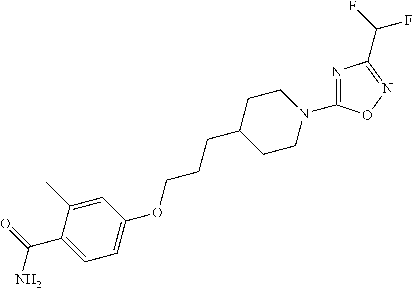 | 4-{3-[1-(3-Difluoromethyl- [1,2,4]oxadiazol-5-yl)- piperidin-4-yl]propoxy}-2- methylbenzamide | RT = 3.52 min; m/z (ES⁺) = 396.15 [M + H]⁺ |
| 42 | 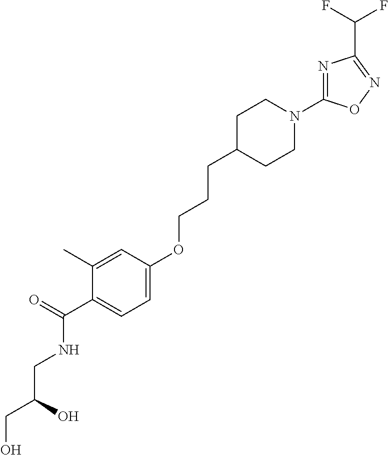 | 4-{3-[1-(3-Difluoromethyl- [1,2,4]oxadiazol-5-yl)- piperidin-4-yl]propoxy}-N- ((R)-2,3-dihydroxypropyl)-2- methylbenzamide | RT = 3.23 min; m/z (ES⁺) = 469.19 [M + H]⁺ |
| 43 | 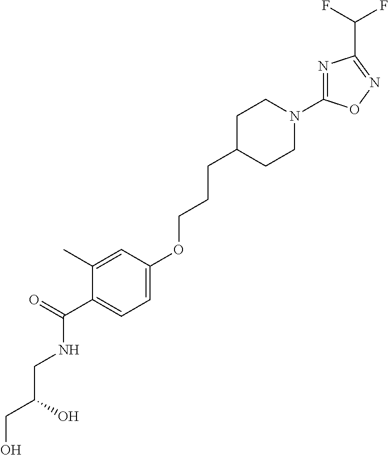 | 4-{3-[1-(3-Difluoromethyl- [1,2,4]oxadiazol-5-yl)- piperidin-4-yl]propoxy}-N- ((S)-2,3-dihydroxypropyl)-2- methylbenzamide | RT = 3.25 min; m/z (ES⁺) = 469.18 [M + H]⁺ |
| 44 | 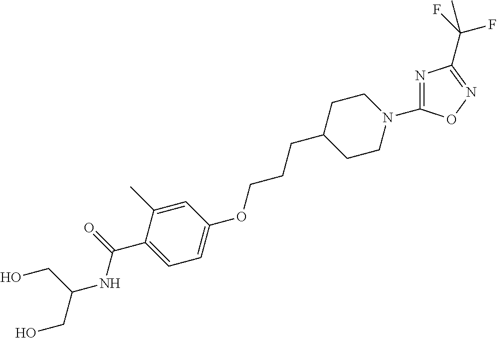 | 4-(3-{1-[3-(1,1-Difluoro- ethyl)-[1,2,4]oxadiazol-5-yl]- piperidin-4-yl}propoxy)-N-(2- hydroxy-1-hydroxymethyl- ethyl)-2-methylbenzamide | RT = 3.38 min; m/z (ES⁺) = 483.25 [M + H]⁺ |
| 45 | 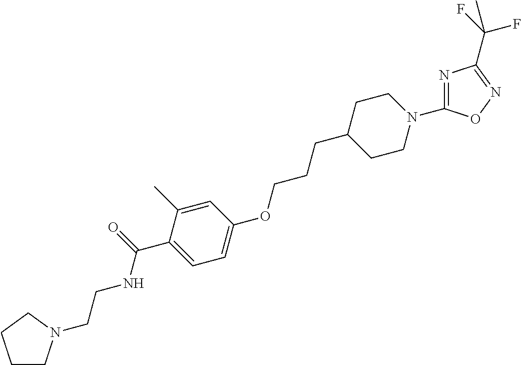 | 4-(3-{1-[3-(1,1-Difluoro- ethyl)-[1,2,4]oxadiazol-5-yl]- piperidin-4-yl}propoxy)-2- methyl-N-(2-pyrrolidin-1-yl- ethyl)benzamide | RT = 2.92 min; m/z (ES⁺) = 506.28 [M + H]⁺ |
| 46 | 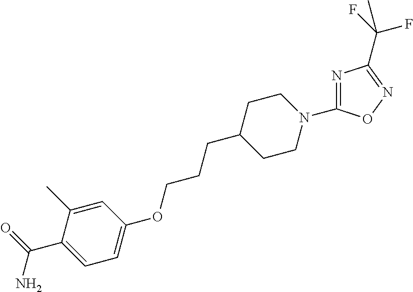 | 4-(3-{1-[3-(1,1-Difluoro- ethyl)-[1,2,4]oxadiazol-5-yl]- piperidin-4-yl}propoxy)-2- methylbenzamide | RT = 3.65 min; m/z (ES⁺) = 410.19 [M + H]⁺ |

The homoenantiomeric amides listed in Table 3 were synthesised by condensing the appropriate acid with the appropriate racemic amine, employing a procedure similar to that outlined in Example 9, followed by resolution of the resulting racemate by preparative chiral HPLC. The preparative chiral HPLC separations used a Daicel Chiralpack IA column (250×20 mm, 5 μm), with an eluent of IH:iPrOH (3:2), at a flow rate of 15 mL/min, and UV detection at 250 nm.

TABLE 3

| Ex | Structure | Name | Spectra: LCMS |
|---|---|---|---|
| 47 | 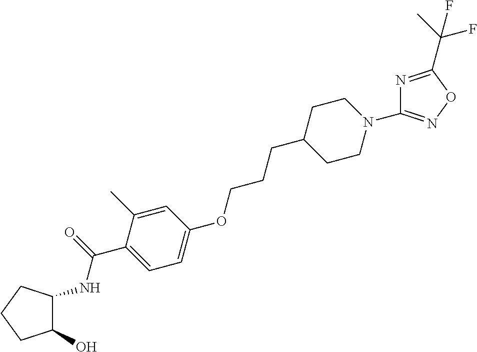 | 4-(3-{1-[5-(1,1-Difluoro- ethyl)-[1,2,4]oxadiazol-3-yl]- piperidin-4-yl}propoxy)-N- ((1S,2S)-2-hydroxy- cyclopentyl)-2-methyl- benzamide | RT = 3.97 min; m/z (ES⁺) = 493.25 [M + H]⁺ |
| 48 | 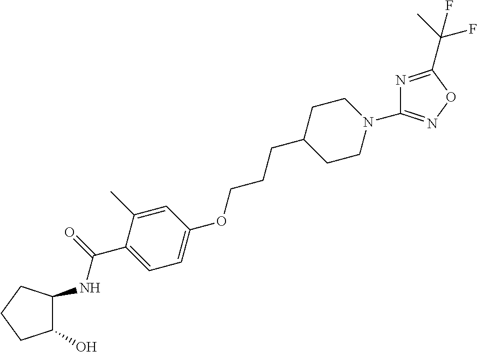 | 4-(3-{1-[5-(1,1-Difluoro- ethyl)-[1,2,4]oxadiazol-3-yl]- piperidin-4-yl}propoxy)-N- ((1R,2R)-2-hydroxy- cyclopentyl)-2-methyl- benzamide | RT = 3.97 min; m/z (ES⁺) = 493.25 [M + H]⁺ |
| 49 | 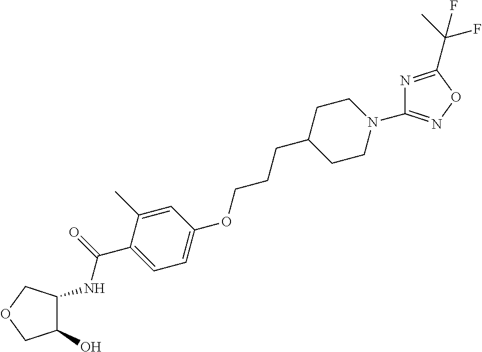 | 4-(3-{1-[5-(1,1-Difluoro- ethyl)-[1,2,4]oxadiazol-3-yl]- piperidin-4-yl}propoxy)-N- ((3S,4R)-4-hydroxytetrahydro- furan-3-yl)-2-methyl- benzamide | RT = 3.73 min; m/z (ES⁺) = 495.23 [M + H]⁺ |
| 50 | 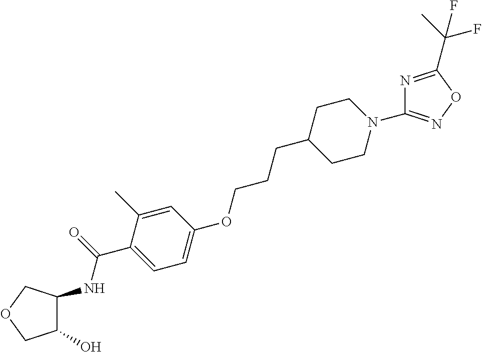 | 4-(3-{1-[5-(1,1-Difluoro- ethyl)-[1,2,4]oxadiazol-3-yl]- piperidin-4-yl}propoxy)-N- ((3R,4S)-4-hydroxytetrahydro- furan-3-yl)-2-methyl- benzamide | RT = 3.73 min; m/z (ES⁺) = 495.23 [M + H]⁺ |
| 51 | 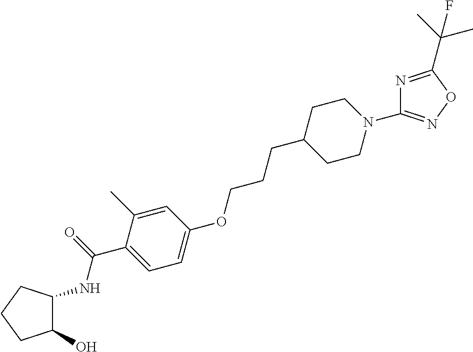 | 4-(3-{1-[5-(1-Fluoro-1-methyl- ethyl)-[1,2,4]oxadiazol-3-yl]- piperidin-4-yl}propoxy)-N- ((1S,2S)-2-hydroxy- cyclopentyl)-2-methyl- benzamide | RT = 3.97 min; m/z (ES⁺) = 489.28 [M + H]⁺ |
| 52 | 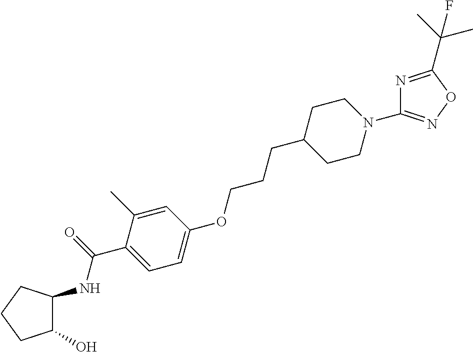 | 4-(3-{1-[5-(1-Fluoro-1-methyl- ethyl)-[1,2,4]oxadiazol-3-yl]- piperidin-4-yl}propoxy)-N- ((1R,2R)-2-hydroxy- cyclopentyl)-2-methyl- benzamide | RT = 3.97 min; m/z (ES⁺) = 489.28 [M + H]⁺ |
| 53 | 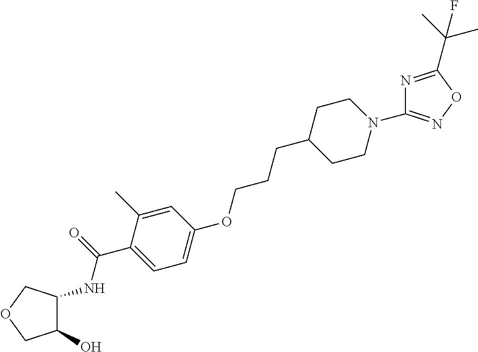 | 4-(3-{1-[5-(1-Fluoro-1-methyl- ethyl)-[1,2,4]oxadiazol-3-yl]- piperidin-4-yl}propoxy)-N- ((3S,4R)-4-hydroxytetrahydro- furan-3-yl)-2-methyl- benzamide | RT = 3.63 min; m/z (ES⁺) = 491.25 [M + H]⁺ |
| 54 | 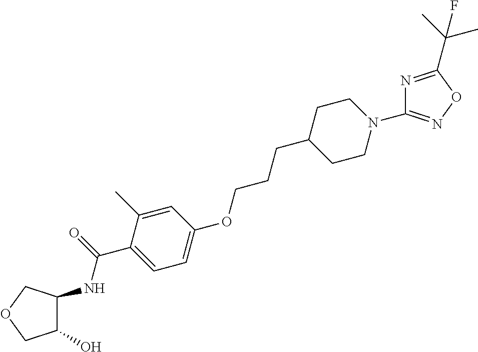 | 4-(3-{1-[5-(1-Fluoro-1-methyl- ethyl)-[1,2,4]oxadiazol-3-yl]- piperidin-4-yl}propoxy)-N- ((3R,4S)-4-hydroxytetrahydro- furan-3-yl)-2-methyl- benzamide | RT = 3.63 min; m/z (ES⁺) = 491.25 [M + H]⁺ |

The amino-containing amides listed in Table 4 were synthesised by condensing the appropriate acid with the appropriate Boc-amino-containing amine, employing procedures similar to that outlined in Example 9 followed by Boc deprotection with HCl in dioxane, employing procedures similar to that outlined in Preparation 2.

TABLE 4

| Ex | Structure | Name | Spectra: LCMS |
|---|---|---|---|
| 55 | 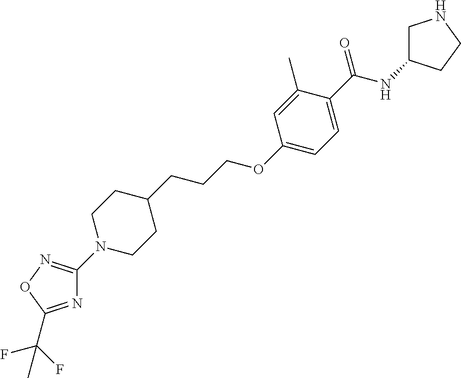 | 4-(3-{1-[5-(1,1-Difluoro- ethyl)-[1,2,4]oxadiazol-3-yl]- piperidin-4-yl}propoxy)-2- methyl-N-(S)-pyrrolidin-3-yl- benzamide | RT = 2.97 min; m/z (ES⁺) = 478.21 [M + H]⁺ |
| 56 | 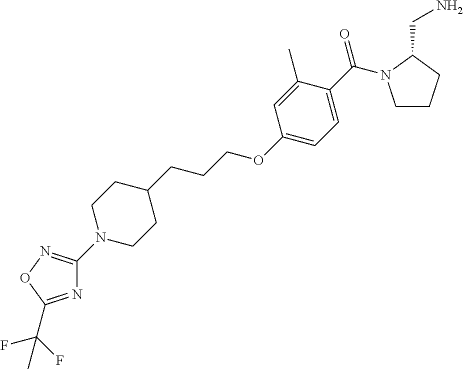 | ((S)-2-Aminomethyl- pyrrolidin-1-yl)-[4-(3-{1-[5- (1,1-difluoroethyl)- [1,2,4]oxadiazol-3-yl]- piperidin-4-yl}propoxy)-2- methylphenyl]methanone | RT = 3.05 min; m/z (ES⁺) = 492.22 [M + H]⁺ |
| 57 | 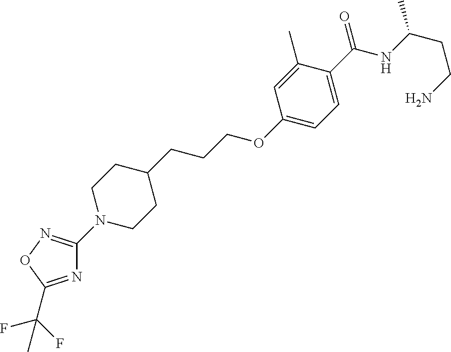 | N-((R)-3-Amino-1-methyl- propyl)-4-(3-{1-[5-(1,1- difluoroethyl)- [1,2,4]oxadiazol-3-yl]- piperidin-4-yl}propoxy)-2- methylbenzamide | RT = 3.00 min; m/z (ES⁺) = 480.22 [M + H]⁺ |
| 58 | 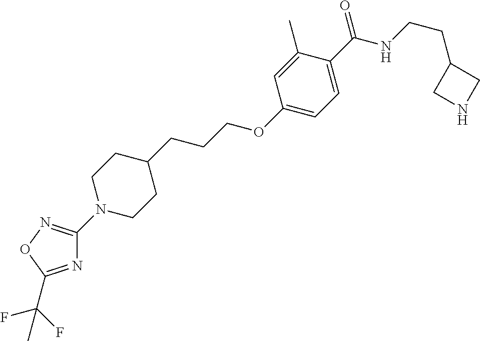 | N-(2-Azetidin-3-ylethyl)-4-(3- {1-[5-(1,1-difluoroethyl)- [1,2,4]oxadiazol-3-yl]- piperidin-4-yl}propoxy)-2- methylbenzamide | RT = 2.93 min; m/z (ES⁺) = 492.22 [M + H]⁺ |
| 59 | 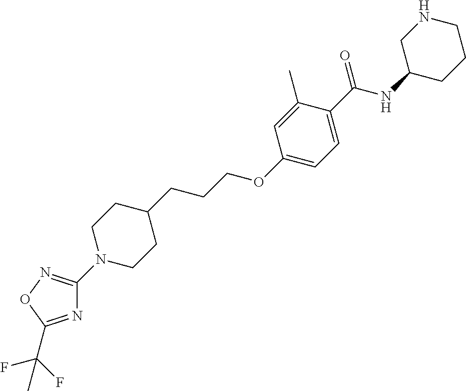 | 4-(3-{1-[5-(1,1-Difluoro- ethyl)-[1,2,4]oxadiazol-3-yl]- piperidin-4-yl}propoxy)-2- methyl-N-(R)-piperidin-3-yl- benzamide | RT = 2.93 min; m/z (ES⁺) = 492.23 [M + H]⁺ |
| 60 | 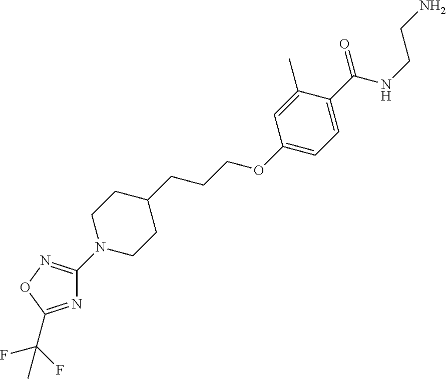 | N-(2-Aminoethyl)-4-(3-{1-[5- (1,1-difluoroethyl)- [1,2,4]oxadiazol-3-yl]- piperidin-4-yl}propoxy)-2- methylbenzamide | RT = 2.92 min; m/z (ES⁺) = 452.20 [M + H]⁺ |
| 61 | 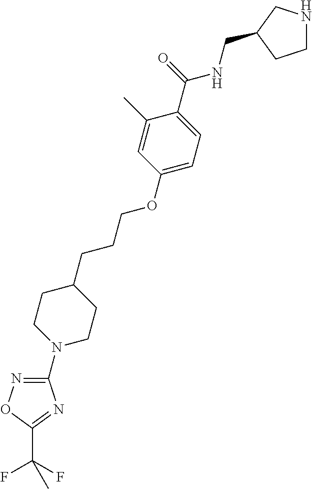 | 4-(3-{1-[5-(1,1-Difluoro- ethyl)-[1,2,4]oxadiazol-3-yl]- piperidin-4-yl}propoxy)-2- methyl-N-(R)-1-pyrrolidin-3- ylmethylbenzamide | RT = 2.88 min; m/z (ES⁺) = 492.22 [M + H]⁺ |
| 62 | 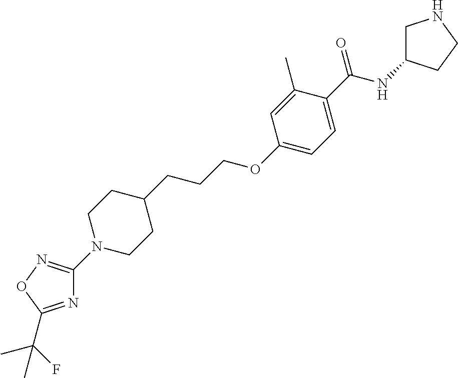 | 4-(3-{1-[5-(1-Fluoro-1-methyl- ethyl)-[1,2,4]oxadiazol-3-yl]- piperidin-4-yl}propoxy)-2- methyl-N-(S)-pyrrolidin-3-yl- benzamide | RT = 2.88 min; m/z (ES⁺) = 474.24 [M + H]⁺ |
| 63 | 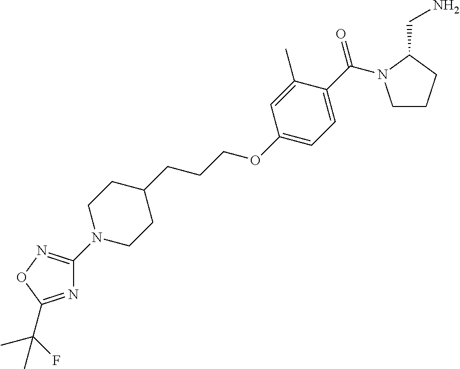 | ((S)-2-Aminomethyl- pyrrolidin-1-yl)-[4-(3-{1-[5-(1- fluoro-1-methylethyl)- [1,2,4]oxadiazol-3-yl]- piperidin-4-yl}propoxy)-2- methylphenyl]methanone | RT = 2.97 min; m/z (ES⁺) = 488.26 [M + H]⁺ |
| 64 | 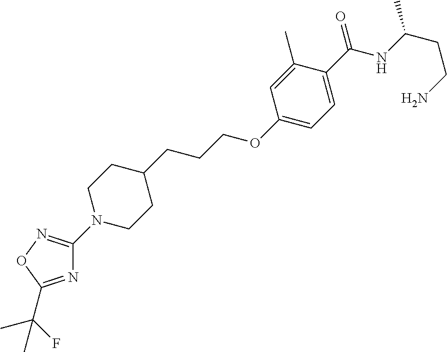 | N-((R)-3-Amino-1-methyl- propyl)-4-(3-{1-[5-(1-fluoro-1- methylethyl)-[1,2,4]oxadiazol- 3-yl]piperidin-4-yl}propoxy)- 2-methylbenzamide | RT = 2.92 min; m/z (ES⁺) = 476.25 [M + H]⁺ |
| 65 | 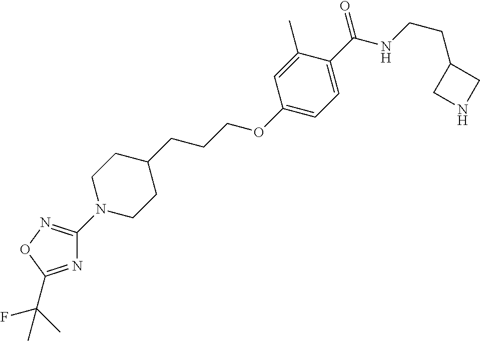 | N-(2-Azetidin-3-ylethyl)-4-(3- {1-[5-(1-fluoro-1-methyl- ethyl)-[1,2,4]oxadiazol-3-yl]- piperidin-4-yl}propoxy)-2- methylbenzamide | RT = 2.85 min; m/z (ES⁺) = 488.26 [M + H]⁺ |
| 66 | 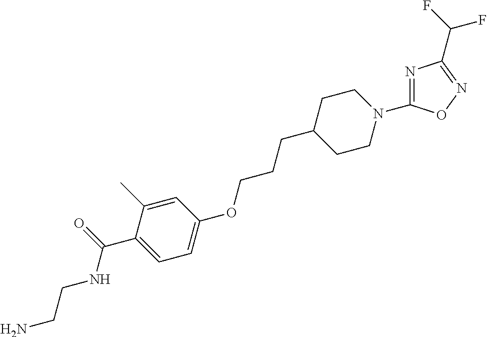 | N-(2-Aminoethyl)-4-{3-[1-(3- difluoromethyl- [1,2,4]oxadiazol-5-yl)- piperidin-4-yl]propoxy}-2- methylbenzamide | RT = 2.62 min; m/z (ES⁺) = 438.20 [M + H]⁺ |
| 67 | 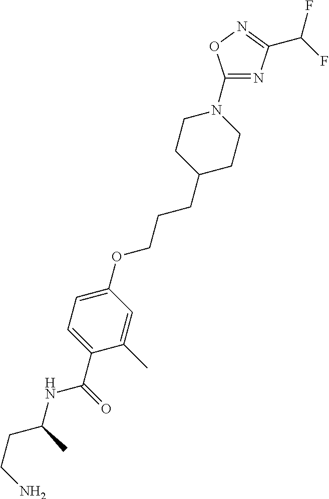 | N-((S)-3-Amino-1-methyl- propyl)-4-{3-[1-(3- difluoromethyl- [1,2,4]oxadiazol-5-yl)- piperidin-4-yl]propoxy}-2- methylbenzamide | RT = 2.73 min; m/z (ES⁺) = 466.23 [M + H]⁺ |
| 68 | 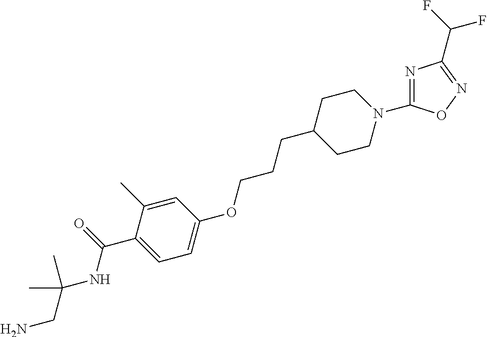 | N-(2-Amino-1,1-dimethyl- ethyl)-4-{3-[1-(3- difluoromethyl- [1,2,4]oxadiazol-5-yl)- piperidin-4-yl]propoxy}-2- methylbenzamide | RT = 2.80 min; m/z (ES⁺) = 466.24 [M + H]⁺ |
| 69 | 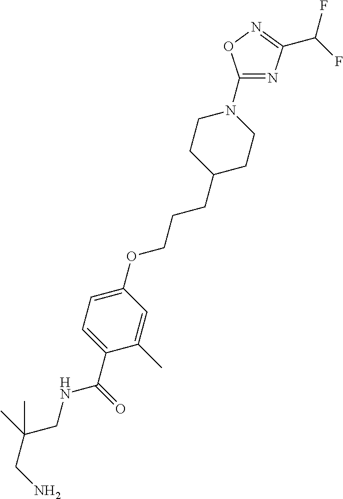 | N-(3-Amino-2,2-dimethyl- propyl)-4-{3-[1-(3- difluoromethyl- [1,2,4]oxadiazol-5-yl)- piperidin-4-yl]propoxy}-2- methylbenzamide | RT = 2.80 min; m/z (ES⁺) = 480.24 [M + H]⁺ |
| 70 | 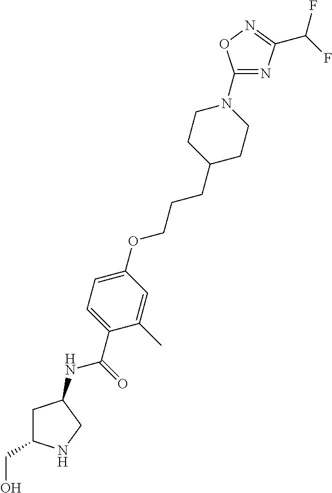 | 4-{3-[1-(3-Difluoromethyl- [1,2,4]oxadiazol-5-yl)- piperidin-4-yl]propoxy}-N- ((3R,5S)-5-hydroxymethyl- pyrrolidin-3-yl)-2-methyl- benzamide | RT = 2.72 min; m/z (ES⁺) = 494.24 [M + H]⁺ |
| 71 | 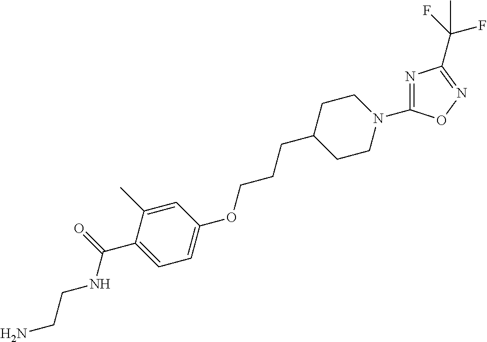 | N-(2-Aminoethyl)-4-(3-{1-[3- (1,1-difluoroethyl)- [1,2,4]oxadiazol-5-yl]- piperidin-4-yl}propoxy)-2- methylbenzamide | RT = 2.72 min; m/z (ES⁺) = 452.17 [M + H]⁺ |
| 72 | 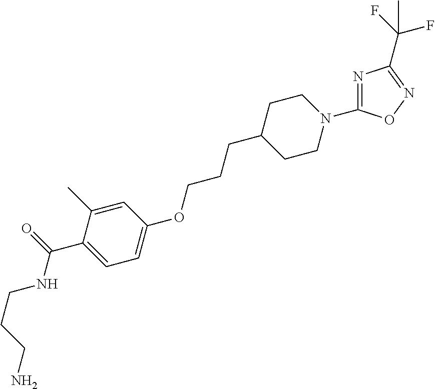 | N-(3-Aminopropyl)-4-(3-{1-[3- (1,1-difluoroethyl)- [1,2,4]oxadiazol-5-yl]- piperidin-4-yl}propoxy)-2- methylbenzamide | RT = 2.79 min; m/z (ES⁺) = 466.18 [M + H]⁺ |
| 73 | 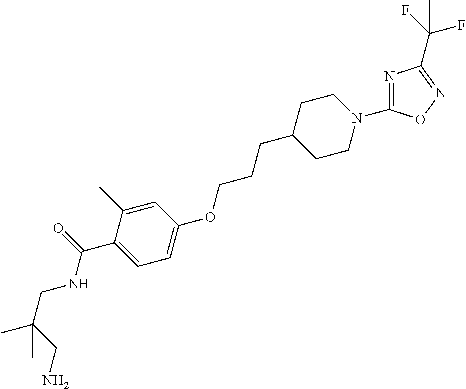 | N-(3-Amino-2,2-dimethyl- propyl)-4-(3-{1-[3-(1,1- difluoroethyl)- [1,2,4]oxadiazol-5-yl]- piperidin-4-yl}propoxy)-2- methylbenzamide | RT = 2.87 min; m/z (ES⁺) = 494.22 [M + H]⁺ |
| 74 | 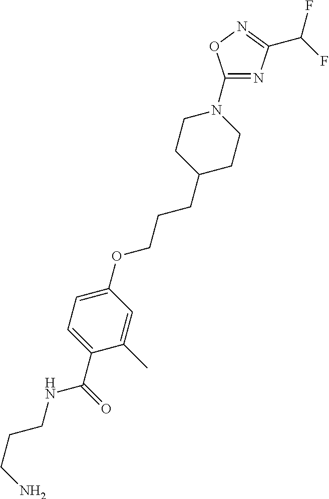 | N-(3-Aminopropyl)-4-{3-[1-(3- difluoromethyl- [1,2,4]oxadiazol-5-yl)- piperidin-4-yl]propoxy}-2- methylbenzamide | RT = 2.63 min; m/z (ES⁺) = 452.19 [M + H]⁺ |

The amides listed in Table 5 were obtained in enantiomerically pure form employing procedures similar to those used for the compounds catalogued in Table 4, with the exception that the individual enantiomers of the Boc-protected intermediates were separated by preparative chiral HPLC using a Daicel Chiralpak IA column (250×20 mm, 5 μm), with an eluent of IH/CHCl₃/iPrOH (7:2:1) at a flow rate of 15 mL/min, and UV detection at 250 nm.

TABLE 5

| Ex | Structure | Name | Spectra: LCMS |
|---|---|---|---|
| 75 | 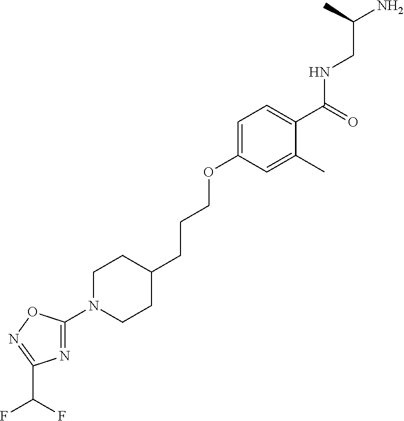 | N-((R)-2-Aminopropyl)-4-{3- [1-(3-difluoromethyl- [1,2,4]oxadiazol-5-yl)- piperidin-4-yl]propoxy}-2- methylbenzamide | RT = 2.67 min; m/z (ES⁺) = 452.21 [M + H]⁺ |
| 76 | 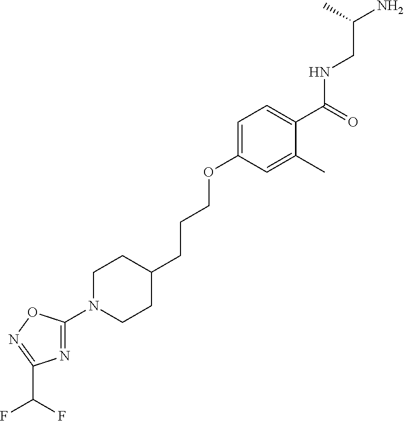 | N-((S)-2-Aminopropyl)-4-{3- [1-(3-difluoromethyl- [1,2,4]oxadiazol-5-yl)- piperidin-4-yl]propoxy}-2- methylbenzamide | RT = 2.67 min; m/z (ES⁺) = 452.21 [M + H]⁺ |
| 77 | 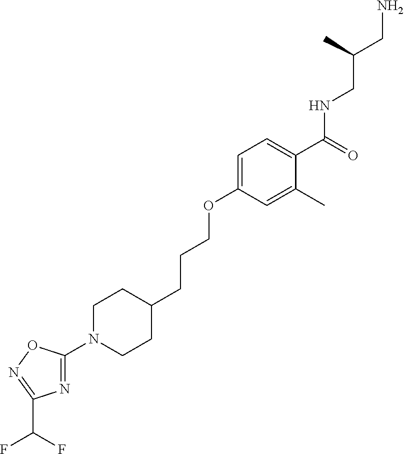 | N-((S)-3-Amino-2-methyl- propyl)-4-{3-[1-(3- difluoromethyl- [1,2,4]oxadiazol-5-yl)- piperidin-4-yl]propoxy}-2- methylbenzamide | RT = 2.73 min; m/z (ES⁺) = 466.22 [M + H]⁺ |
| 78 | 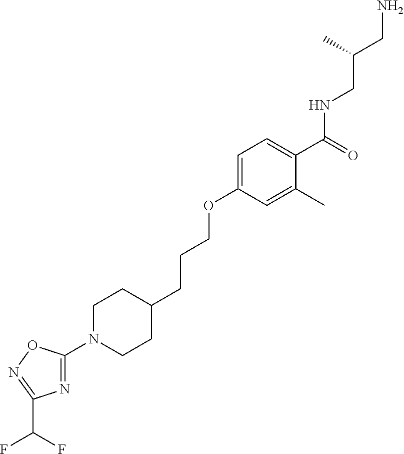 | N-((R)-3-Amino-2-methyl- propyl)-4-{3-[1-(3- difluoromethyl- [1,2,4]oxadiazol-5-yl)- piperidin-4-yl]propoxy}-2- methylbenzamide | RT = 2.73 min; m/z (ES⁺) = 466.22 [M + H]⁺ |
| 79 | 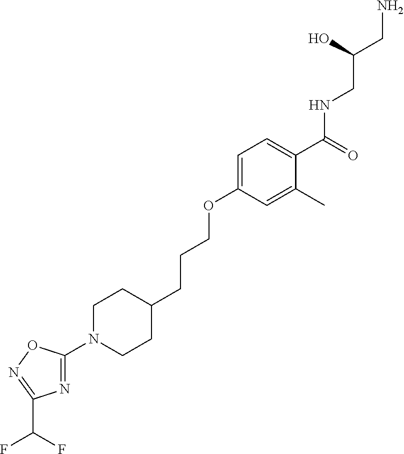 | N-((S)-3-Amino-2-hydroxy- propyl)-4-{3-[1-(3- difluoromethyl- [1,2,4]oxadiazol-5-yl)- piperidin-4-yl]propoxy}-2- methylbenzamide | RT = 2.59 min; m/z (ES⁺) = 468.19 [M + H]⁺ |
| 80 | 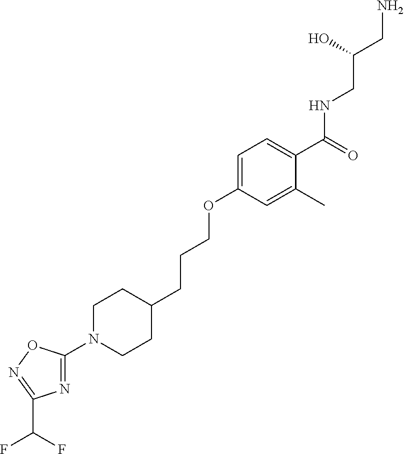 | N-((R)-3-Amino-2-hydroxy- propyl)-4-{3-[1-(3- difluoromethyl- [1,2,4]oxadiazol-5-yl)- piperidin-4-yl]propoxy}-2- methylbenzamide | RT = 2.59 min; m/z (ES⁺) = 468.19 [M + H]⁺ |

### Example 81

### (3-{4-[(R)-3-(4-Methanesulfonylphenoxy)-1-methylpropyl]piperidin-1-yl}-[1,2,4]oxadiazol-5-ylmethyl)dimethylamine

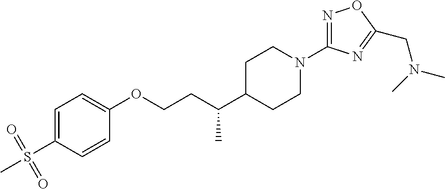

HOBt.H₂O (81.0 mg, 600 μmol), EDCI (114 mg, 600 μmol) and DIPEA (78.0 mg, 600 μmol) were added to a stirred solution of dimethylaminoacetic acid (56.0 mg, 542 μmol) in DMF (2 mL). After stirring at ambient temperature for 15 min, N-hydroxy-4-[(R)-3-(4-methanesulfonylphenoxy)-1-methylpropyl]piperidine-1-carboxamidine (Preparation 8, 200 mg, 542 μmol) was added and the resulting mixture was stirred at ambient temperature for 38 h. The solvent was removed in vacuo and the remainder was diluted with EtOAc, washed with saturated aqueous Na₂CO₃ solution, water and brine, before being dried (MgSO₄). Filtration, solvent evaporation and purification by column chromotography (EtOAc) provided the title compound: RT=2.56 min; m/z (ES⁺)=436.96 [M+H]⁺.

The compounds listed in Table 6 were synthesised by condensing the appropriate acid with N-hydroxy-4-[(R)-3-(4-methanesulfonylphenoxy)-1-methylpropyl]piperidine-1-carboxamidine (Preparation 8), employing a procedure similar to that outlined in Example 81.

TABLE 6

| Ex | Structure | Name | Spectra: LCMS |
|---|---|---|---|
| 82 | 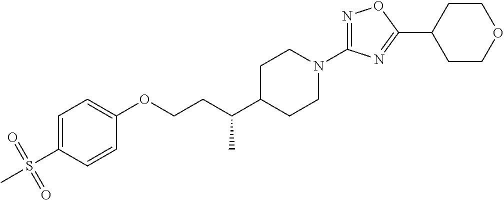 | 4-[(R)-3-(4-Methanesulfonyl- phenoxy)-1-methylpropyl]-1- [5-(tetrahydropyran-4-yl)- [1,2,4]oxadiazol-3-yl]- piperidine | RT = 3.85 min; m/z (ES⁺) = 464.18 [M + H]⁺ |
| 83 | 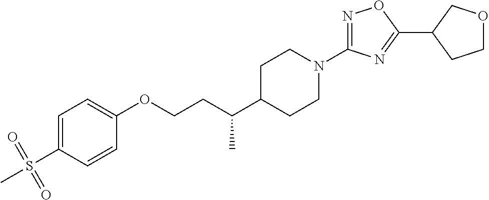 | 4-[(R)-3-(4-Methanesulfonyl- phenoxy)-1-methylpropyl]-1- [5-(tetrahydrofuran-3-yl)- [1,2,4]oxadiazol-3-yl]- piperidine | RT = 3.78 min; m/z (ES⁺) = 450.18 [M + H]⁺ |
| 84 | 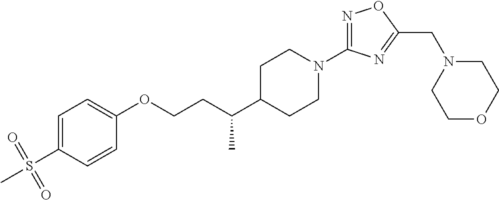 | 4-(3-{4-[(R)-3-(4- Methanesulfonylphenoxy)-1- methylpropyl]piperidin-1-yl}- [1,2,4]oxadiazol-5-ylmethyl)- morpholine | RT = 3.30 min; m/z (ES⁺) = 479.11 [M + H]⁺ |
| 85 | 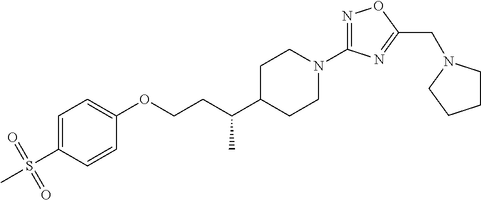 | 4-[(R)-3-(4-Methanesulfonyl- phenoxy)-1-methylpropyl]-1- (5-pyrrolidin-1-ylmethyl- [1,2,4]oxadiazol-3-yl)- piperidine | RT = 2.75 min; m/z (ES⁺) = 463.18 [M + H]⁺ |
| 86 | 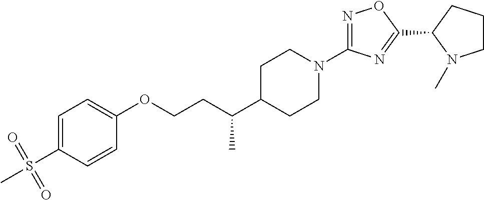 | 4-[(R)-3-(4-Methanesulfonyl- phenoxy)-1-methylpropyl]-1- [5-((S)-1-methylpyrrolidin-2- yl)-[1,2,4]oxadiazol-3-yl]- piperidine | RT = 2.68 min; m/z (ES⁺) = 463.15 [M + H]⁺ |

### Example 87

### 4-[(R)-3-(4-Methanesulfonylphenoxy)-1-methylpropyl]-1-(3-methoxymethyl-[1,2,4]oxadiazol-5-yl)piperidine

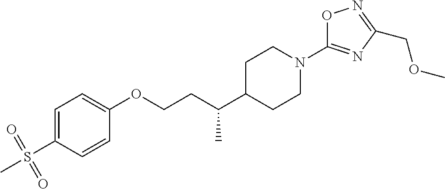

ZnCl₂ (1M in Et₂O, 357 μL, 357 μmol) was added to a stirred solution of 4-[(R)-3-(4-methanesulfonylphenoxy)-1-methylpropyl]piperidine-1-carbonitrile (Preparation 7, 100 mg, 297 μmol) and N-hydroxy-2-methoxyacetamidine (37.0 mg, 357 μmol) in EtOAc (3 mL) and THF (3 mL). The reaction mixture was stirred an ambient temperature for 72 h before removing the solvents in vacuo. The remainder was dissolved in EtOH (10 mL) and 12M HCl (1 mL), before being heated at 70° C. for 16 h. The reaction mixture was concentrated to one half of the original volume and adjusted to pH 8 with saturated aqueous NaHCO₃ solution. The mixture was extracted with Et₂O (3×), and the combined organic extracts were washed with brine and dried (MgSO₄). Filtration, solvent evaporation and purification by column chromatography (EtOAc—IH, 1:1 to 7:3) afforded the title compound: RT=3.52 min; m/z (ES⁺)=423.94 [M+H]⁺.

### Example 88

### 2-(5-{4-[(R)-3-(4-Methanesulfonylphenoxy)-1-methylpropyl]piperidin-1-yl}-[1,2,4]oxadiazol-3-yl)ethanol

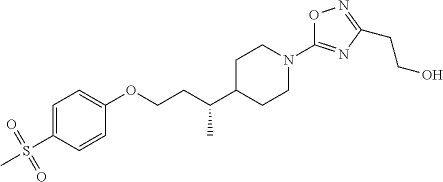

The title compound was prepared from 4-[(R)-3-(4-methanesulfonylphenoxy)-1-methyl-propyl]piperidine-1-carbonitrile (Preparation 7, 100 mg, 297 μmol) and 3,N-dihydroxy-propionamidine (37.0 mg, 357 μmol) using a procedure similar to that outlined in Example 87: RT=3.19 min; m/z (ES⁺)=423.94 [M+H]⁺.

### Example 89

### 4-[(R)-3-(4-Methanesulfonylphenoxy)-1-methylpropyl]-1-(3-pyrrolidin-1-ylmethyl-[1,2,4]oxadiazol-5-yl)piperidine

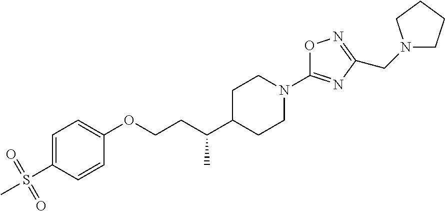

Pyrrolidine (77.0 μL, 920 μmol) was added to a stirred solution of 1-(3-chloromethyl-[1,2,4]oxadiazol-5-yl)-4-{(R)-3-(4-methanesulfonylphenoxy)-1-methylpropyl}piperidine (Preparation 9, 113 mg, 260 μmol) in DMF (1 mL) and the resulting solution was stirred at ambient temperature for 72 h. Further pyrrolidine (22.0 μL, 260 μmol) was added and the solution was heated at 40° C. for 1 h. The reaction mixture was poured into H₂O (50 mL), extracted with EtOAc (2×100 mL), then the combined organic extracts were washed with brine, dried (MgSO₄), filtered and concentrated in vacuo. Purification by column chromatography (EtOAc-MeOH—NEt₃, 93:6:1) afforded the title compound: RT=2.75 min; m/z (ES⁺)=463.15 [M+H]⁺.

### Example 90

### (5-{4-[(R)-3-(4-Methanesulfonylphenoxy)-1-methylpropyl]piperidin-1-yl}-[1,2,4]oxadiazol-3-ylmethyl)dimethylamine

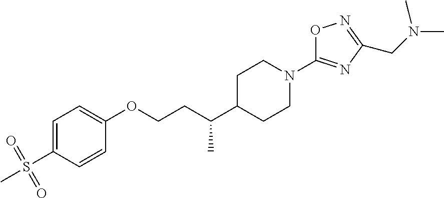

The title compound was synthesised from 1-(3-chloromethyl-[1,2,4]oxadiazol-5-yl)-4-[(R)-3-(4-methanesulfonylphenoxy)-1-methylpropyl]piperidine (Preparation 9, 208 mg, 490 μmol) and dimethylamine (2.44 mL, 4.86 mmol) employing a procedure similar to that outlined in Example 89: RT=2.88 min; m/z (ES⁺)=437.11 [M+H]⁺.

### Example 91

### 4-[(R)-3-(4-Methanesulfonylphenoxy)-1-methylpropyl]-1-(5-pyrrolidin-2-yl-[1,2,4]oxadiazol-3-yl)piperidine

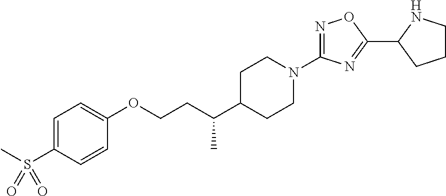

2-(3-{4-[(R)-3-(4-Methanesulfonylphenoxy)-1-methylpropyl]piperidin-1-yl}-[1,2,4]oxadiazol-5-yl)pyrrolidine-1-carboxylic acid tert-butyl ester (Preparation 13, 815 mg, 1.49 mmol) in 4M HCl in dioxane was stirred at ambient temperature for 1 h before concentrating the reaction mixture in vacuo. The residue was partitioned between DCM (100 mL) and saturated aqueous NaHCO₃ solution (100 mL). The organic layer was separated, washed with saturated aqueous NaHCO₃ solution (2×50 mL) and brine, dried (MgSO₄), filtered and concentrated in vacuo. Purification by column chromatography afforded the title compound: RT=2.63 min; m/z (ES⁺)=449.16 [M+H]⁺.

### Example 92

### 4-{3-[1-(5-Cyclobutyl-[1,2,4]oxadiazol-3-yl)piperidin-4-yl]propoxy}-N—((R)-2-hydroxy-1-methylethyl)-2-methylbenzamide

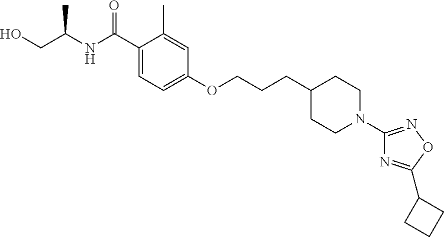

A solution of cyclobutanecarbonyl chloride (32.6 mg, 275 μmol), 4-{3-[1-(N-hydroxycarbamimidoyl)piperidin-4-yl]propoxy}-N—((R)-2-hydroxy-1-methylethyl)-2-methyl-benzamide (Preparation 20, 100 mg, 255 μmol), and NEt₃ (52 μL, 375 μmol) in DCE (4 mL) was stirred at 20° C. for 2 h, before being heated to 80° C. for 2.5 h. On cooling, the reaction was partitioned between CH₂Cl₂ (8 mL) and H₂O (8 mL). The aqueous phase was further extracted with CH₂Cl₂ (2 mL), then the combined organic extracts were shaken with MP-carbonate resin. The resin was then filtered off, washing with CH₂Cl₂ (2×2 mL), then the filtrate was concentrated and the residue purified by preparative HPLC to furnish the title compound: RT=3.73 min; ink (ES⁺)=457.21 [M+H]⁺.

The compounds listed in Table 7 were synthesised by condensing the appropriate carboxylic acid with the appropriate amidoxime, employing procedures similar to that outlined in Example 92.

TABLE 7

| Ex | Structure | Name | Spectra: LCMS |
|---|---|---|---|
| 93 | 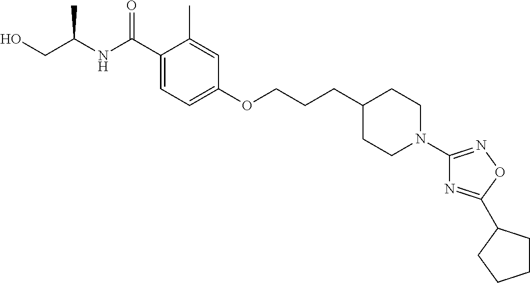 | 4-{3-[1-(5-Cyclopentyl- [1,2,4]oxadiazol-3-yl)piperidin-4- yl]propoxy}-N-((R)-2-hydroxy-1- methylethyl)-2-methylbenzamide | RT = 3.95 min; m/z (ES⁺) = 471.23 [M + H]⁺ |
| 94 | 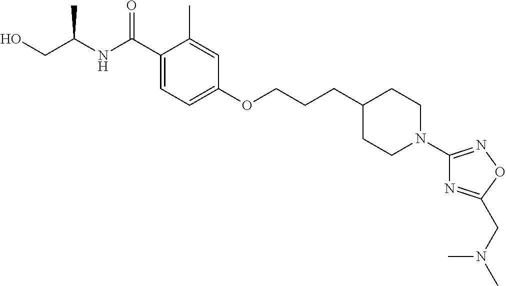 | 4-{3-[1-(5-Dimethylamino- methyl-[1,2,4]oxadiazol-3- yl)piperidin-4-yl]propoxy}-N- ((R)-2-hydroxy-1-methylethyl)-2- methylbenzamide | RT = 2.42 min; m/z (ES⁺) = 460.22 [M + H]⁺ |
| 95 | 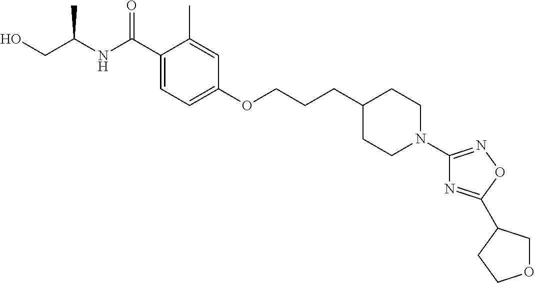 | N-((R)-2-Hydroxy-1-methyl- ethyl)-2-methyl-4-(3-{1-[5- (tetrahydrofuran-3-yl)- [1,2,4]oxadiazol-3-yl]piperidin-4- yl}propoxy)benzamide | RT = 3.30 min; m/z (ES⁺) = 473.22 [M + H]⁺ |
| 96 | 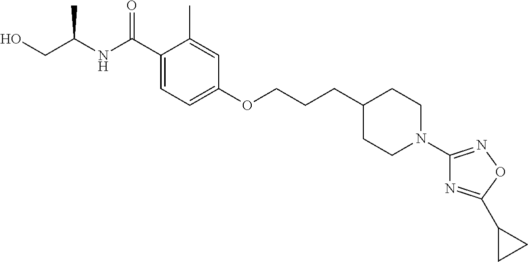 | 4-{3-[1-(5-Cyclopropyl- [1,2,4]oxadiazol-3-yl)piperidin-4- yl]propoxy}-N-((R)-2-hydroxy-1- methylethyl)-2-methylbenzamide | RT = 3.52 min; m/z (ES⁺) = 443.24 [M + H]⁺ |
| 97 | 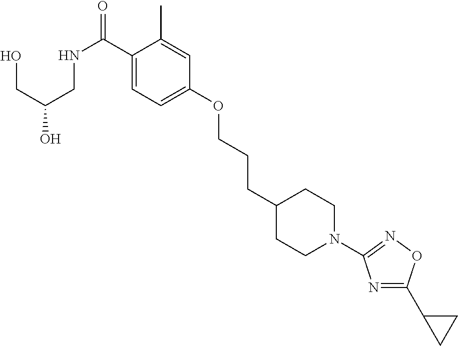 | 4-{3-[1-(5-Cyclopropyl- [1,2,4]oxadiazol-3-yl)piperidin-4- yl]propoxy}-N-((S)-2,3- dihydroxypropyl)-2-methyl- benzamide | RT = 3.29 min; m/z (ES⁺) = 459.24 [M + H]⁺ |
| 98 | 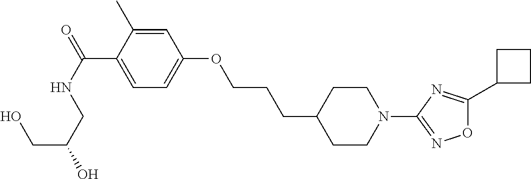 | 4-{3-[1-(5-Cyclobutyl- [1,2,4]oxadiazol-3-yl)piperidin-4- yl]propoxy}-N-((S)-2,3- dihydroxypropyl)-2-methyl- benzamide | RT = 3.48 min; m/z (ES⁺) = 473.27 [M + H]⁺ |
| 99 | 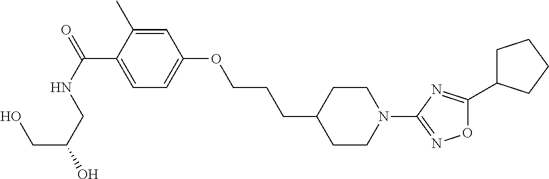 | 4-{3-[1-(5-Cyclopentyl- [1,2,4]oxadiazol-3-yl)piperidin-4- yl]propoxy}-N-((S)-2,3- dihydroxypropyl)-2-methyl- benzamide | RT = 3.67 min; m/z (ES⁺) = 487.28 [M + H]⁺ |
| 100 | 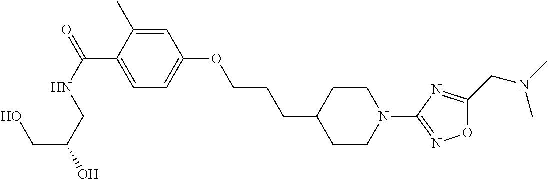 | N-((S)-2,3-Dihydroxypropyl)-4- {3-[1-(5-dimethylaminomethyl- [1,2,4]oxadiazol-3-yl)piperidin-4- yl]propoxy}-2-methylbenzamide | RT = 2.29 min; m/z (ES⁺) = 476.25 [M + H]⁺ |
| 101 | 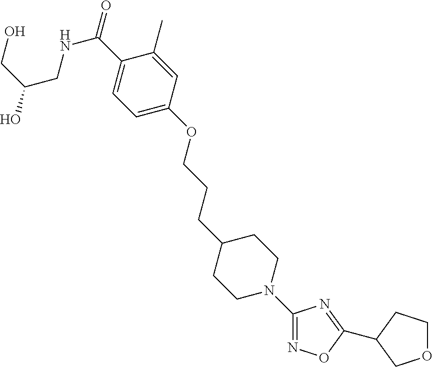 | N-((S)-2,3-Dihydroxypropyl)-2- methyl-4-(3-{1-[5-(tetrahydro- furan-3-yl)-[1,2,4]oxadiazol-3- yl]piperidin-4-yl}propoxy)- benzamide | RT = 3.09 min; m/z (ES⁺) = 489.23 [M + H]⁺ |

### Example 102

### 4-{3-[1-(5-Cyclopropylmethyl-[1,2,4]oxadiazol-3-yl)piperidin-4-yl]propoxy}-N—((R)-2-hydroxy-1-methylethyl)-2-methylbenzamide

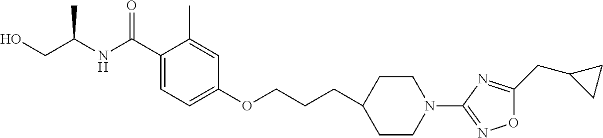

A solution of HOBt.H₂O (44 mg, 288 μmol) in DMF (1 mL) was added to a stirred solution of cyclopropylacetic acid (27.5 mg, 275 μmol) and 4-{3-[1-(N-hydroxycarbamimidoyl)piperidin-4-yl]propoxy}-N—((R)-2-hydroxy-1-methylethyl)-2-methyl-benzamide (Preparation 20, 100 mg, 255 μmol) in DMF (2 mL). The mixture was treated with a solution of EDCI (62 mg, 325 μmol) in DMF (1.5 mL), then stiffing was continued at 20° C. for 3 h, before being heated at 80° C. for 3 h. On cooling, the reaction was partitioned between CH₂Cl₂ (9 mL) and H₂O (9 mL). The aqueous phase was further extracted with CH₂Cl₂ (3 mL), then the combined organic extracts were concentrated and the residue purified by preparative HPLC to furnish the title compound: RT=3.68 min; m/z (ES⁺)=457.26 [M+H]⁺.

The compounds listed in Table 8 were synthesised by condensing the appropriate carboxylic acid with the appropriate amidoxime, employing a procedure similar to that outlined in Example 102.

TABLE 8

| | | | Spectra: |
|---|---|---|---|
| Ex | Structure | Name | LCMS |
| 103 | 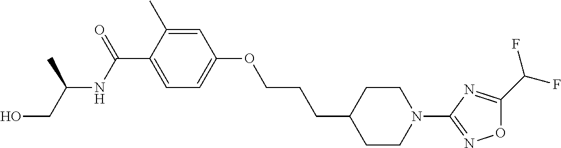 | 4-{3-[1-(5-Difluoromethyl- [1,2,4]oxadiazol-3-yl) piperidin-4-yl]propoxy}- N-((R)-2-hydroxy-1- methylethyl)-2-methyl- benzamide | RT = 3.60 min; m/z (ES⁺) = 453.21 [M + H]⁺ |
| 104 | 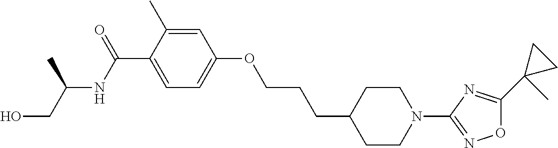 | N-((R)-2-Hydroxy-1- methylethyl)- 2-methyl-4-(3-{1-[5- (1-methylcyclopropyl)- [1,2,4]oxadiazol-3-yl] piperidin-4- yl}propoxy)benzamide | RT = 3.67 min; m/z (ES⁺) = 457.23 [M + H]⁺ |
| 105 | 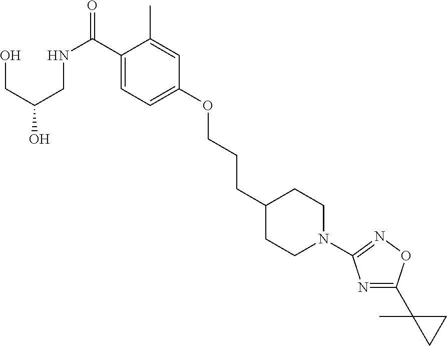 | N-((S)-2,3-Dihydroxy- propyl)-2-methyl- 4-(3-{1-[5-(1-methyl- cyclopropyl)-[1,2,4]oxa- diazol-3-yl] piperidin-4-yl}propoxy)- benzamide | RT = 3.48 min; m/z (ES⁺) = 473.23 [M + H]⁺ |
| 106 | 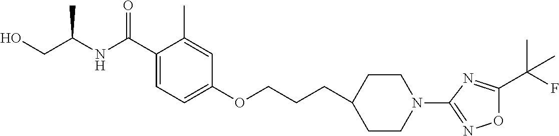 | 4-(3-{1-[5-(1-Fluoro-1- methylethyl)- [1,2,4]oxadiazol-3-yl]- piperidin-4-yl}propoxy)-N- ((R)-2-hydroxy-1-methyl- ethyl)-2-methylbenzamide | RT = 3.75 min; m/z (ES⁺) = 463.24 [M + H]⁺ |
| 107 | 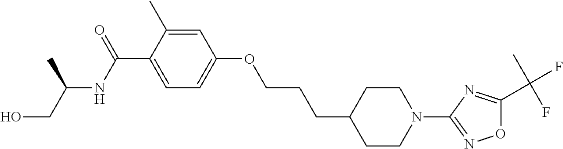 | 4-(3-{1-[5-(1,1-Di- fluoroethyl)- [1,2,4]oxadiazol-3-yl] piperidin-4-yl}propoxy)- N-((R)-2-hydroxy-1- methylethyl)-2-methyl- benzamide | RT = 3.82 min; m/z (ES⁺) = 467.24 [M + H]⁺ |
| 108 | 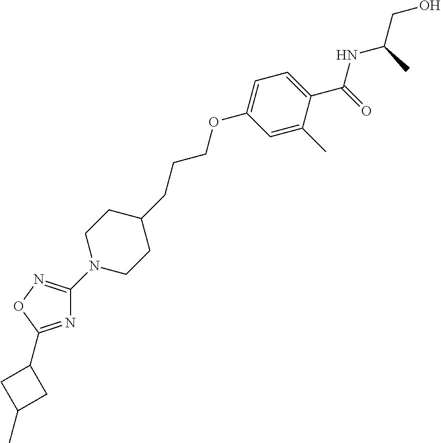 | N-((R)-2-Hydroxy-1- methylethyl)- 2-methyl-4-(3-{1-[5-(3- methylcyclobutyl)-[1,2,4] oxadiazol-3-yl]piperidin-4- yl}propoxy)benzamide | RT = 4.03 min; m/z (ES⁺) = 471.28 [M + H]⁺ |
| 109 | 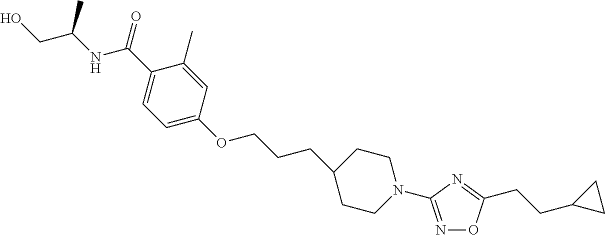 | 4-(3-{1-[5-(2-Cyclo- propylethyl)-[1,2,4]oxa- diazol-3-yl]piperidin-4- yl}propoxy)-N-((R)-2- hydroxy-1-methylethyl)- 2-methylbenzamide | RT = 3.90 min; m/z (ES⁺) = 471.26 [M + H]⁺ |
| 110 | 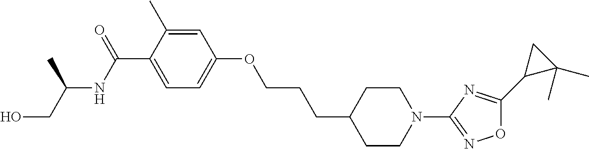 | 4-(3-{1-[5-(2,2-Dimethyl- cyclopropyl)-[1,2,4] oxadiazol-3-yl]piperidin-4- yl}propoxy)-N-((R)-2- hydroxy-1-methylethyl)-2- methylbenzamide | RT = 3.95 min; m/z (ES⁺) = 471.28 [M + H]⁺ |
| 111 | 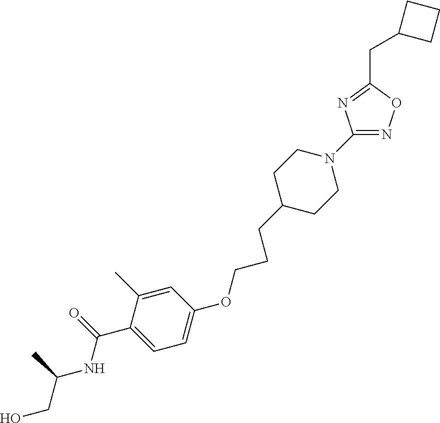 | 4-{3-[1-(5-Cyclo- butylmethyl- [1,2,4]oxadiazol-3-yl) piperidin-4-yl]propoxy}- N-((R)-2-hydroxy-1- methylethyl)-2-methyl- benzamide | RT = 3.97 min; m/z (ES⁺) = 471.28 [M + H]⁺ |
| 112 | 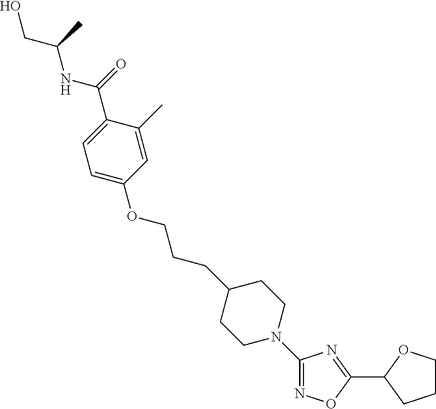 | N-((R)-2-Hydroxy- 1-methylethyl)- 2-methyl-4-(3-{1-[5- (tetrahydrofuran-2-yl)- [1,2,4]oxadiazol-3-yl] piperidin-4- yl}propoxy)benzamide | RT = 3.47 min; m/z (ES⁺) = 473.26 [M + H]⁺ |
| 113 | 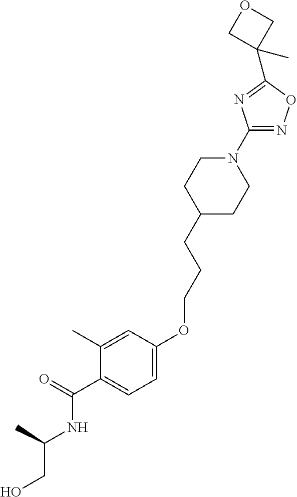 | N-((R)-2-Hydroxy-1- methylethyl)- 2-methyl-4-(3-{1-[5- (3-methyloxetan-3-yl)- [1,2,4]oxadiazol-3-yl] piperidin-4- yl}propoxy)benzamide | RT = 3.40 min; m/z (ES⁺) = 473.25 [M + H]⁺ |
| 114 | 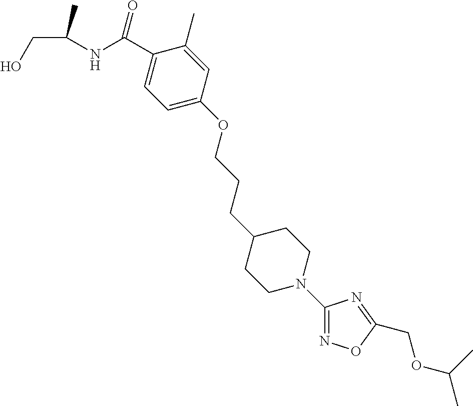 | N-((R)-2-Hydroxy-1- methylethyl)- 4-{3-[1-(5- isopropoxymethyl- [1,2,4]oxadiazol-3-yl) piperidin-4-yl]propoxy}- 2-methylbenzamide | RT = 3.60 min; m/z (ES⁺) = 475.27 [M + H]⁺ |
| 115 | 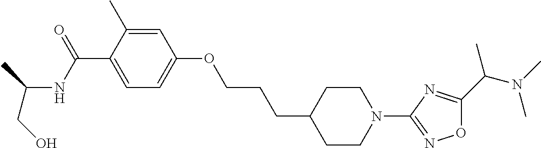 | 4-(3-{1-[5-(1-Di- methylamino-ethyl)- [1,2,4]oxadiazol-3-yl]- piperidin-4-yl}prop- oxy)-N-((R)-2-hydroxy-1- methylethyl)-2- methylbenzamide | RT = 2.40 min; m/z (ES⁺) = 474.29 [M + H]⁺ |
| 116 | 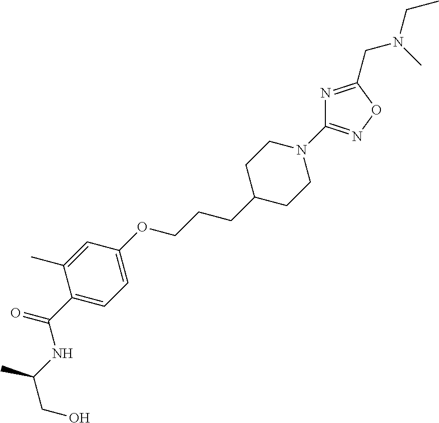 | 4-[3-(1-{5-[(Ethylmethyl- amino)-methyl]-[1,2,4]oxa- diazol-3-yl}-piperidin-4- yl)propoxy]-N-((R)-2- hydroxy-1-methylethyl)-2- methylbenzamide | RT = 2.43 min; m/z (ES⁺) = 474.29 [M + H]⁺ |
| 117 | 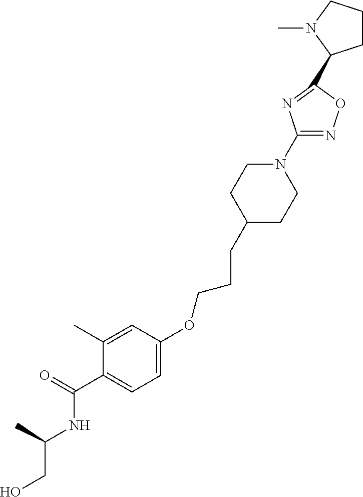 | N-((R)-2-Hydroxy-1- methylethyl)- 2-methyl-4-(3-{1-[5- ((S)-1-methylpyrrolidin-2- yl)-[1,2,4]oxadiazol-3-yl] piperidin-4- yl}propoxy)benzamide | RT = 2.42 min; m/z (ES⁺) = 486.29 [M + H]⁺ |
| 118 | 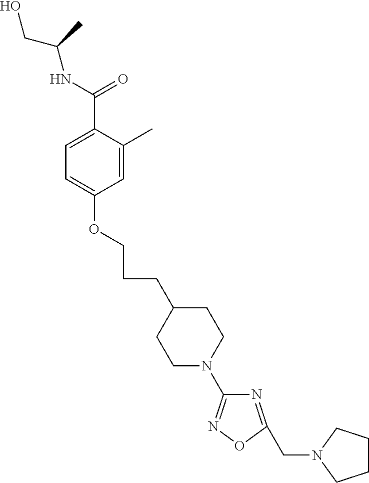 | N-((R)-2-Hydroxy-1- methylethyl)- 2-methyl-4-{3-[1-(5- pyrrolidin-1-ylmethyl- [1,2,4]oxadiazol-3-yl) piperidin-4- yl]propoxy}benzamide | RT = 2.38 min; m/z (ES⁺) = 486.29 [M + H]⁺ |
| 119 | 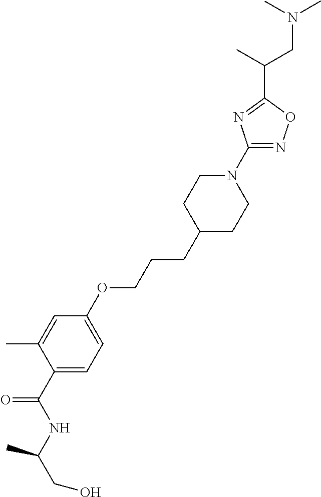 | 4-(3-{1-[5-(2-Dimethyl- amino-1-methylethyl)- [1,2,4]oxadiazol-3- yl]piperidin-4-yl} propoxy)-N-((R)-2- hydroxy- 1-methylethyl)-2- methylbenzamide | RT = 2.42 min; m/z (ES⁺) = 488.30 [M + H]⁺ |
| 120 | 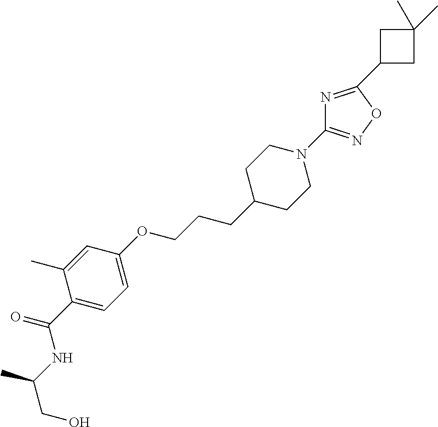 | 4-(3-{1-[5-(3,3-Dimethyl- cyclobutyl)-[1,2,4]oxa- diazol-3-yl]piperidin-4- yl}propoxy)-N-((R)-2- hydroxy-1-methylethyl)-2- methylbenzamide | RT = 4.22 min; m/z (ES⁺) = 485.30 [M + H]⁺ |
| 121 | 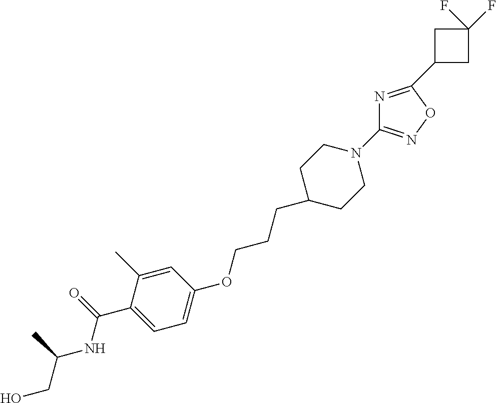 | 4-(3-{1-[5-(3,3-Difluoro- cyclobutyl)-[1,2,4]oxa- diazol-3-yl]piperidin-4- yl}propoxy)-N-((R)-2- hydroxy-1-methylethyl)-2- methylbenzamide | RT = 3.72 min; m/z (ES⁺) = 493.25 [M + H]⁺ |
| 122 | 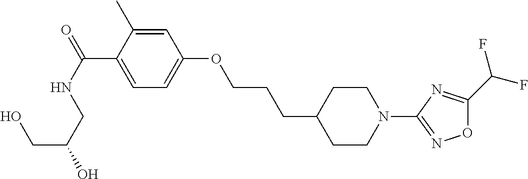 | 4-{3-[1-(5-Difluoromethyl- [1,2,4]oxadiazol-3-yl) piperidin-4-yl]propoxy}- N-((S)-2,3-dihydroxy- propyl)-2-methyl- benzamide | RT = 3.40 min; m/z (ES⁺) = 469.19 [M + H]⁺ |
| 123 | 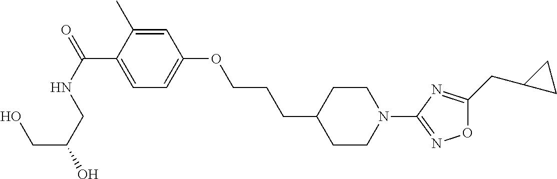 | 4-{3-[1-(5-Cyclopropyl- methyl-[1,2,4]oxadiazol-3- yl)piperidin-4- yl]propoxy}-N-((S)-2,3-di- hydroxypropyl)-2-methyl- benzamide | RT = 3.52 min; m/z (ES⁺) = 473.32 [M + H]⁺ |
| 124 | 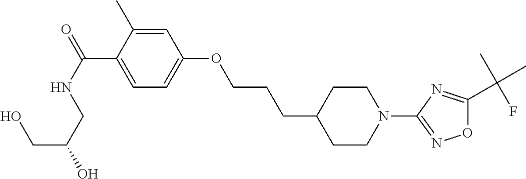 | N-((S)-2,3-Dihydroxy- propyl)-4-(3-{1-[5- (1-fluoro-1-methylethyl)- [1,2,4]oxadiazol-3-yl]- piperidin-4-yl}propoxy)-2- methylbenzamide | RT = 3.38 min; m/z (ES⁺) = 479.22 [M + H]⁺ |
| 125 | 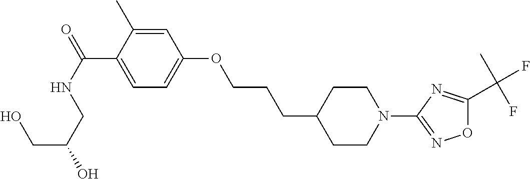 | 4-(3-{1-[5-(1,1-Difluoro- ethyl)-[1,2,4]oxadiazol- 3-yl]piperidin-4- yl}propoxy)-N-((S)-2,3-di- hydroxypropyl)-2-methyl- benzamide | RT = 3.52 min; m/z (ES⁺) = 483.18 [M + H]⁺ |
| 126 | 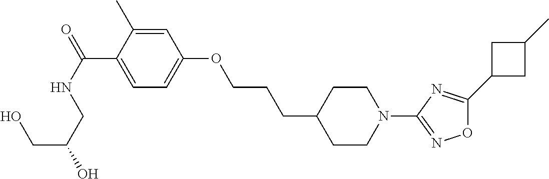 | N-((S)-2,3-Dihydroxy- propyl)-2-methyl-4-(3- {1-[5-(3-methyl- cyclobutyl)-[1,2,4]oxa- diazol-3-yl] piperidin-4-yl}propoxy)- benzamide | RT = 3.72 min; m/z (ES⁺) = 487.22 [M + H]⁺ |
| 127 | 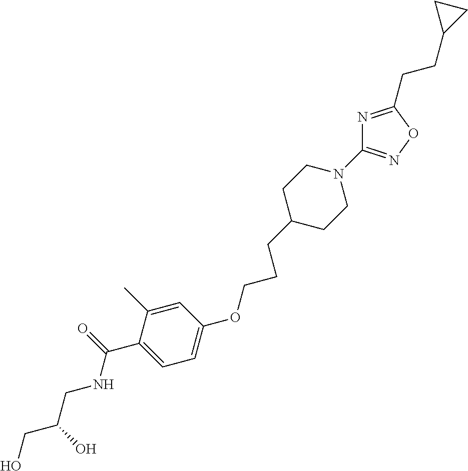 | 4-(3-{1-[5-(2-Cyclo- propylethyl)-[1,2,4]oxa- diazol-3-yl]piperidin-4- yl}propoxy)-N-((S)-2,3-di- hydroxypropyl)-2-methyl- benzamide | RT = 3.50 min; m/z (ES⁺) = 487.22 [M + H]⁺ |
| 128 | 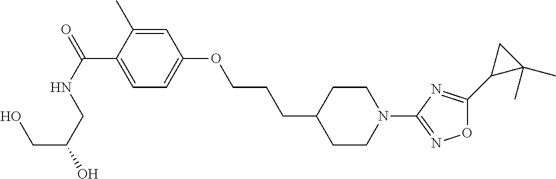 | N-((S)-2,3-Dihydroxy- propyl)-4- (3-{1-[5-(2,2-dimethyl- cyclopropyl)- [1,2,4]oxadiazol-3- yl]piperidin-4-yl}propoxy)- 2-methylbenzamide | RT = 3.60 min; m/z (ES⁺) = 487.22 [M + H]⁺ |
| 129 | 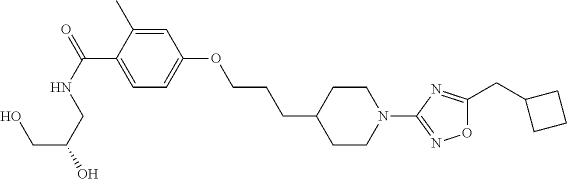 | 4-{3-[1-(5-Cyclo- butylmethyl-[1,2,4]oxa- diazol-3-yl)piperidin-4- yl]propoxy}-N-((S)-2,3- dihydroxypropyl)-2-methyl- benzamide | RT = 3.67 min; m/z (ES⁺) = 487.22 [M + H]⁺ |
| 130 | 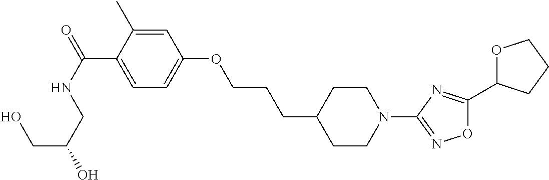 | N-((S)-2,3-Dihydroxy- propyl)-2-methyl-4-(3- {1-[5-(tetrahydro-furan- 2-yl)-[1,2,4]oxadiazol-3- yl]piperidin-4-yl} propoxy)-benzamide | RT = 3.22 min; m/z (ES⁺) = 489.21 [M + H]⁺ |
| 131 | 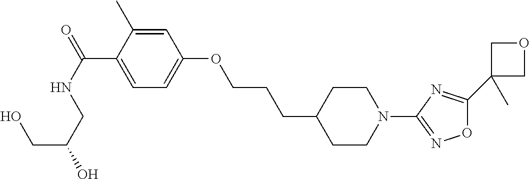 | N-((S)-2,3-Dihydroxy- propyl)-2-methyl-4-(3- {1-[5-(3-methyl-oxetan- 3-yl)-[1,2,4]oxadiazol-3- yl]piperidin-4-yl}propoxy)- benzamide | RT = 3.10 min; m/z (ES⁺) = 489.21 [M + H]⁺ |
| 132 | 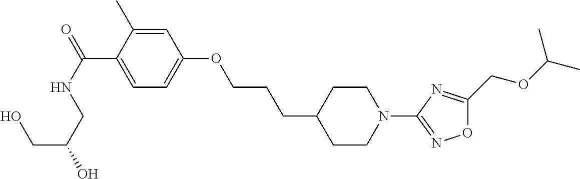 | N-((S)-2,3-Dihydroxy- propyl)-4-{3-[1-(5-iso- propoxymethyl- [1,2,4]oxadiazol-3-yl) piperidin-4-yl]propoxy}- 2-methylbenzamide | RT = 3.35 min; m/z (ES⁺) = 491.23 [M + H]⁺ |
| 133 | 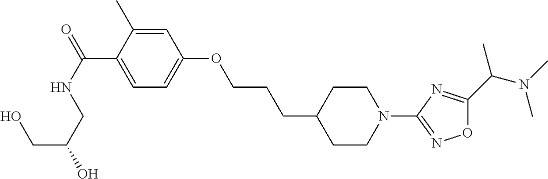 | N-((S)-2,3-Dihydroxy- propyl)-4- (3-{1-[5-(1-dimethylamino- ethyl)-[1,2,4]oxadiazol-3- yl]-piperidin-4-yl} propoxy)- 2-methylbenzamide | RT = 2.43 min; m/z (ES⁺) = 490.29 [M + H]⁺ |
| 134 | 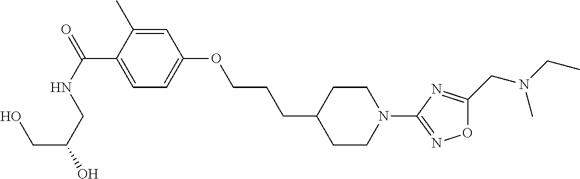 | N-((S)-2,3-Dihydroxy- propyl)-4-[3-(1-{5- [(ethylmethylamino)-meth- yl]-[1,2,4]oxadiazol-3-yl}- piperidin-4-yl)propoxy]-2- methylbenzamide | RT = 2.32 min; m/z (ES⁺) = 490.29 [M + H]⁺ |
| 135 | 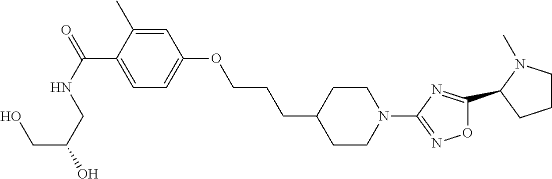 | N-((S)-2,3-Dihydroxy- propyl)-2-methyl-4-(3-{1- [5-((S)-1-methyl- pyrrolidin- 2-yl)-[1,2,4]oxadiazol- 3-yl]piperidin-4-yl} propoxy)-benzamide | RT = 2.35 min; m/z (ES⁺) = 502.29 [M + H]⁺ |
| 136 | 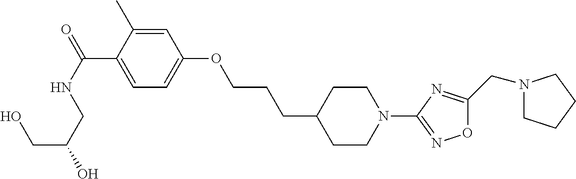 | N-((S)-2,3-Dihydroxy- propyl)-2-methyl-4-{3-[1- (5-pyrrolidin-1-ylmethyl- [1,2,4]oxadiazol-3-yl)- piperidin-4-yl]propoxy}- benzamide | RT = 2.45 min; m/z (ES⁺) = 502.29 [M + H]⁺ |
| 137 | 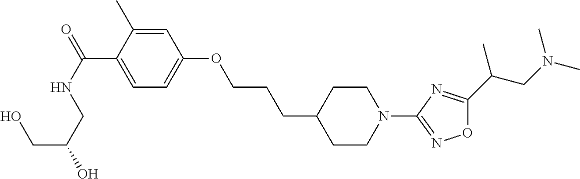 | N-((S)-2,3-Dihydroxy- propyl)-4-(3-{1-[5-(2- dimethylamino-1- methylethyl)-[1,2,4]oxa- diazol-3-yl] piperidin-4-yl}propoxy)-2- methylbenzamide | RT = 2.43 min; m/z (ES⁺) = 504.30 [M + H]⁺ |
| 138 | 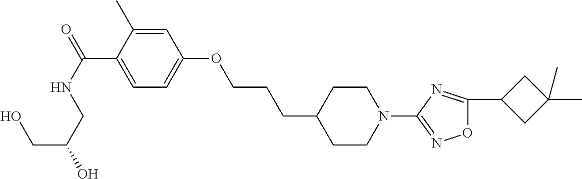 | N-((S)-2,3-Dihydroxy- propyl)-4-(3-{1-[5- (3,3-dimethyl-cyclobutyl)- [1,2,4]oxadiazol-3-yl] piperidin-4-yl}propoxy)-2- methylbenzamide | RT = 3.88 min; m/z (ES⁺) = 501.24 [M + H]⁺ |
| 139 | 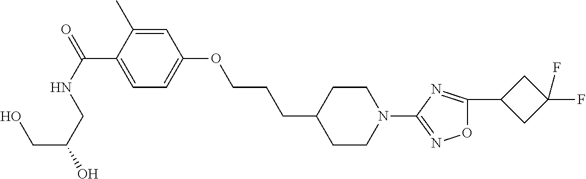 | 4-(3-{1-[5-(3,3-Difluoro- cyclobutyl)-[1,2,4] oxadiazol-3-yl]piperidin- 4-yl}propoxy)-N- ((S)-2,3-dihydroxypropyl)- 2-methylbenzamide | RT = 3.38 min; m/z (ES⁺) = 509.20 [M + H]⁺ |
| 140 | 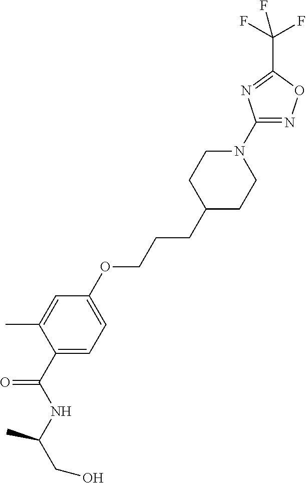 | N-((R)-2-Hydroxy-1- methyl- ethyl)-2-methyl-4-{3-[1-(5- trifluoromethyl-[1,2,4] oxadiazol-3-yl)piperidin-4- yl]propoxy}-benzamide | RT = 4.00 min; m/z (ES⁺) = 471.23 [M + H]⁺ |
| 141 | 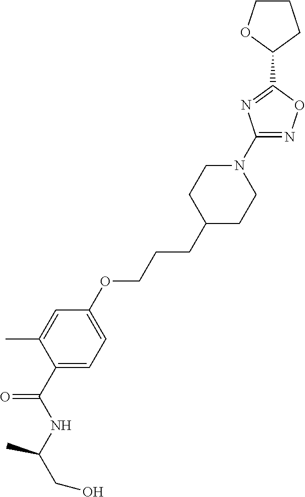 | N-((R)-2-Hydroxy-1- methyl- ethyl)-2-methyl-4-(3-{1- [(R)-5-(tetrahydrofuran-2- yl)-[1,2,4]oxadiazol-3-yl] piperidin- 4-yl}propoxy)benzamide | RT = 3.42 min; m/z (ES⁺) = 473.26 [M + H]⁺ |
| 142 | 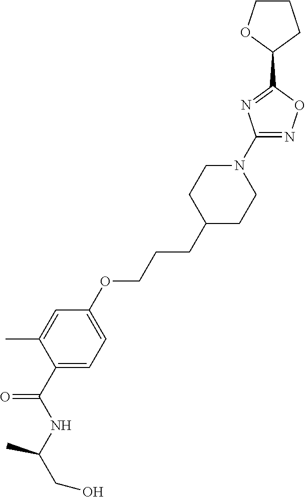 | N-((R)-2-Hydroxy-1- methyl- ethyl)-2-methyl-4-(3-{1- [(S)-5-(tetrahydrofuran- 2-yl)-[1,2,4]oxadiazol-3- yl]piperidin- 4-yl}propoxy)benzamide | RT = 3.43 min; m/z (ES⁺) = 473.26 [M + H]⁺ |

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Step 3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line may be used to investigate the effect of compounds of the invention on intracellular levels of cyclic AMP (cAMP). The cell monolayers are washed with phosphate buffered saline and stimulated at 37° C. for 30 min with various concentrations of compound in stimulation buffer plus 1% DMSO. Cells are then lysed and cAMP content determined using the Perkin Elmer AlphaScreen™ (Amplified Luminescent Proximity Homogeneous Assay) cAMP kit. Buffers and assay conditions are as described in the manufacturer's protocol.

### In Vivo Feeding Study

The effect of compounds of the invention on body weight and food and water intake may be examined in freely-feeding male Sprague-Dawley rats maintained on reverse-phase lighting. Test compounds and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage. Animals are maintained on a reverse phase light-dark cycle (lights off for 8 h from 09.30-17.30 h) during which time the room was illuminated by red light Animals have free access to a standard powdered rat diet and tap water during a two week acclimatization period. The diet is contained in glass feeding jars with aluminum lids. Each lid had a 3-4 cm hole in it to allow access to the food Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 μl lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 ml/well and cultured for 3 days and the medium then discarded. Cells are washed ×2 with supplemented Krebs-Ringer buffer (KRB) containing 119 mM NaCl, 4.74 mM KCl, 2.54 mM CaCl₂, 1.19 mM MgSO₄, 1.19 mM KH2PO4, 25 mM NaHCO₃, 10 mM HEPES at pH 7.4 and 0.1% bovine serum albumin. Cells are incubated with 1 ml KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 uM) are then added to duplicate wells in 1 ml KRB, supplemented with 5.6 mM glucose. After 30 min incubation at 37° C. samples are removed for determination of insulin levels. Measurement of insulin is done using the Mercodia Rat insulin ELISA kit, following the manufacturers instructions, with a standard curve of known insulin concentrations. For each well insulin levels are corrected by subtraction of the basal secretion level from the pre-incubation in the absence of glucose. Data was analysed using XLfit 3 software.

### Oral Glucose Tolerance Tests

The effects of compounds of the invention on oral glucose (Glc) tolerance were evaluated in male Sprague-Dawley rats. Food was withdrawn 16 h before administration of Glc and remained withdrawn throughout the study. Rats had free access to water during the study. A cut was made to the animals' tails, then blood (1 drop) was removed for measurement of basal Glc levels 60 min before administration of the Glc load. Then, the rats were weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples were then taken from the cut tip of the tail 5, 15, 30, 60, 120, and 180 min after Glc administration. Blood glucose levels were measured just after collection using a commercially available glucose-meter (OneTouch® Ultra™ from Lifescan). Representative compounds of the invention statistically reduced the Glc excursion at doses of ≦10 mg kg⁻¹.

The effects of compounds of the invention on oral glucose (Glc) tolerance may also evaluated in male C57B1/6 or male ob/ob mice. Food is withdrawn 5 h before administration of Glc and remained withdrawn throughout the study. Mice have free access to water during the study. A cut is made to the animals' tails, then blood (20 μL) is removed for measurement of basal Glc levels 45 min before administration of the Glc load. Then, the mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) are then taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at rt for 30 min before being read against Glc standards (Sigma glucose/urea nitrogen combined standard set).

1. A compound of formula (I), or a pharmaceutically acceptable salt thereof: 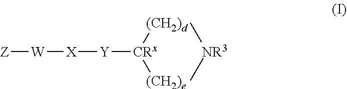
wherein Z is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR¹, CN, NO₂, —(CH₂)ⱼ—S(O)ₘR¹, —(CH₂)ⱼ—C(O)NR¹R¹¹, NR¹R¹¹, NR²C(O)R¹, NR²C(O)NR¹R¹¹, NR²SO₂R¹, SO₂NR¹R¹¹, C(O)R², C(O)OR², —P(O)(CH₃)₂, —(CH₂)ⱼ-(4- to 7-membered heterocyclyl) or —(CH₂)ⱼ-(5- to 6-membered heteroaryl);
m is 0, 1 or 2;
j is 0, 1 or 2;
W and Y are independently a bond, an unbranched or a branched C₁₋₄ alkylene optionally substituted by hydroxy or C₁₋₃ alkoxy, or an unbranched or a branched C₂₋₄ alkenylene;
X is selected from CH₂, O, S, CH(OH), CH(halogen), CF₂, C(O), C(O)O, C(O)S, SC(O), C(O)CH₂S, C(O)CH₂C(OH), C(OH)CH₂C(O), C(O)CH₂C(O), OC(O), NR⁵, CH(NR⁵R⁵⁵), C(O)NR², NR²C(O), S(O) and S(O)₂;
R^{x} is hydrogen or hydroxy;
R¹ and R¹¹ are independently hydrogen, C₁₋₅ alkyl, which may optionally be substituted by halo, hydroxy, Ci₋₄ alkoxy-, aryloxy-, arylC₁₋₄ alkoxy-, C₁₋₄ alkylS(O)—, C₃₋₇ heterocyclyl, C(O)OR⁷ or N(R²)₂; or may be C₃₋₇ cycloalkyl or heterocyclyl, wherein the cyclic groups may be substituted with one or more substituents selected from halo, C₁₋₄ alkyl, C₁₋₄ fluoroalkyl, OR⁶, UN, SO₂CH₃, CH₂OH, N(R²)₂ and NO₂; or taken together R¹ and R¹¹ may form a 5- or 6-membered heterocyclic ring optionally substituted by hydroxy, C₁₋₄ alkyl, C₁₋₄ hydroxyalkyl, or CH₂NH₂ and optionally containing a further heteroatom selected from O and NR²; or R¹¹ is C₁₋₄ alkyloxy-;
R² are independently hydrogen or C₁₋₄ alkyl; or a group N(R²)₂ may form a 4- to 7-membered heterocyclic ring optionally containing a further heteroatom selected from O and NR²;
R³ is: 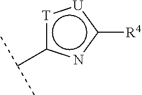
wherein one of T and U is O and the other is N;
R⁴ is C₁₋₃ hydroxyalkyl, C₁₋₃ alkoxyC₁₋₃ alkyl, C₁₋₃ fluoroalkyl, —(C₁₋₃alkylene)ₖ-N(R⁶)₂, —(C₁₋₃alkylene)ₖ-C₃₋₆ cycloalkyl or —(C₁₋₃ alkylene)ₖ-4- to 6-membered heterocyclyl where the cycloalkyl and heterocyclyl groups may be optionally substituted with one or more C₁₋₃ alkyl or fluorine groups;
k is 0 or 1;
R⁵ and R⁵⁵ are independently hydrogen or C₁₋₄ alkyl; or taken together R⁵ and R⁵⁵ may form a 5- or 6-membered heterocyclic ring; or a group NR⁵ may represent NS(O)₂-(2-NO₂—C₆H₄);
R⁶ are independently selected from hydrogen and C₁₋₃ alkyl;
R⁷ is hydrogen or C₁₋₄ alkyl;
d is 0, 1, 2 or 3; and
e is 1, 2, 3, 4 or 5, provided that d+e is 2, 3, 4 or 5.

2. A compound according to claim 1, or a pharmaceutically acceptable salt thereof, wherein Z represents phenyl or a 6-membered heteroaryl group containing up to two N heteroatoms substituted as defined in claim 1.

3. A compound according to claim 2, or a pharmaceutically acceptable salt thereof, wherein Z represents phenyl substituted as defined in claim 1.

4. A compound according, to claim 3, or a pharmaceutically acceptable salt thereof, wherein Z is substituted by —SO₂Me or —CONHR^{d}, wherein R^{d} is hydrogen, 5-membered heterocyclyl, C₁₋₃ alkyl, or C₂₋₃ alkyl substituted by amino or one or two hydroxy groups, and wherein Z is optionally additionally substituted by one or two methyl groups.

5. A compound according to claim 1, or a pharmaceutically acceptable salt thereof, wherein —W—X—Y— is —O—CH₂—CH₂—CR^{y}—, where R^{y} is hydrogen or methyl.

6. A compound according to claim 1, or a pharmaceutically acceptable salt thereof, wherein d and e represent 2.

7. A compound according to claim 1, or a pharmaceutically acceptable salt thereof, wherein R^{x} is hydrogen.

8. A compound according to claim 1, or a pharmaceutically acceptable salt thereof, wherein R⁴ is C₂₋₅ alkyl.

9. A compound of formula (Ia), or a pharmaceutically acceptable salt thereof 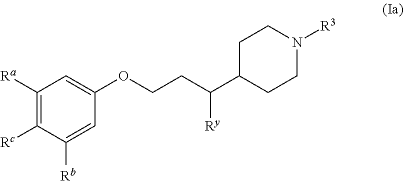
wherein:
R³ is as described in claim 1;
R^{y} is hydrogen or methyl;
R^{a} and R^{b} are independently selected from hydrogen and methyl;
R^{c} is —SO₂Me or —CONHR^{d};
R^{d} is hydrogen, 5-membered. heterocyclyl, C₁₋₃ alkyl, or C₂₋₃ alkyl substituted by amino or one or two hydroxy groups.

10. A compound of claim 1, wherein the compound is any one of Examples 1 to 142, or a pharmaceutically acceptable salt thereof.

11. A pharmaceutical composition comprising a compound according to claim 1, or a pharmaceutically acceptable salt thereof and a pharmaceutically acceptable carrier.

12. A method for the treatment of a disease or condition in which GPR119 plays a role comprising a step of administering to a subject in need thereof an effective amount of a compound according to claim 1, or a pharmaceutically acceptable salt thereof

13. A method for the regulation of satiety comprising a step of administering to a subject in need thereof an effective amount of a compound according to claim 1, or a pharmaceutically acceptable salt thereof.

14-19. (canceled)

20. The method of claim 12, wherein the disease or condition in which GPR119 plays a role is obesity.

21. The method of claim 12, wherein the disease or condition in which GPR119 plays a role is diabetes.

22. The method of claim 12, wherein the disease or condition in which GPR119 plays a role is metabolic syndrome (syndrome X), impaired glucose tolerance, hyperlipidemia, hypertriglyceridemia, hypercholesterolemia, low HDL levels, or hypertension.
